(19) United States
(12) Patent Application Publication
(10) Pub. No.: US 2016/0160255 A1
(43) Pub. Date: Jun. 9, 2016

(54) Methods and Materials for Producing 6-Carbon Monomers

(71) Applicant: INVISTA North America S.á.r.l., Wilmington, DE (US)

(72) Inventors: Adriana Leonora Botes, Rosedale East (GB); Alex Van Eck Conradie, Eaglescliffe (GB); Nadia Kadi, Middlesbrough (GB)

(21) Appl. No.: 14/941,501

(22) Filed: Nov. 13, 2015

Related U.S. Application Data
(60) Provisional application No. 62/079,903, filed on Nov. 14, 2014.
(60) Provisional application No. 62/255,276, filed on Nov. 13, 2015.

(51) Int. Cl. C12P 19/32; C12P 7/44; C12P 13/00; C12P 17/10; C12P 7/18; C07F 9/6561; C12N 9/02; C07C 229/08; C07C 225/06; C07C 47/19; C07C 233/31; C07C 47/12; C12P 7/42; C12N 9/10

(52) U.S. Cl. CPC C12P 19/32; C12P 7/42; C12P 7/44; C12P 13/001; C12P 17/10; C12P 7/18; C12N 9/1029; C12N 9/1096; C12N 9/0008; C07C 229/08; C07C 225/06; C07C 47/19; C07C 233/31; C07C 47/12; C07F 9/65616; C12Y 203/01016; C12Y 203/01174; C12Y 206/01; C12Y 102/99006

(57) ABSTRACT

This document describes biochemical pathways for producing 6-hydroxyhexanoic acid using a polypeptide having β-ketothiolase activity to form a 3-oxo-6-hydroxyhexanoyl-CoA intermediate. 6-hydroxyhexanoic acid can be enzymatically converted to adipic acid, caprolactam, 6-aminohexanoic acid, hexamethylenediamine or 1,6-hexanediol. This document also describes recombinant hosts producing 6-hydroxyhexanoic acid as well as adipic acid, caprolactam, 6-aminohexanoic acid, hexamethylenediamine and 1,6-hexanediol.

### CROSS-REFERENCE TO RELATED APPLICATIONS

This application claims the benefit of U.S. Application Nos. 62/079,903, filed on Nov. 14, 2014, and 62/255,276, filed Nov. 13, 2015, the disclosures of which are incorporated by reference herein in their entireties.

### TECHNICAL FIELD

This invention provides non-naturally occurring methods for producing 6 carbon monomers. The invention provides biosynthesizing 3-oxo-6-hydroxyhexanoyl-CoA using a polypeptide having β-ketothiolase activity, and enzymatically converting 3-oxo-6-hydroxyhexanoyl-CoA to 6-hydroxyhexanoic acid using one or more of a polypeptide having 3-hydroxyacyl-CoA dehydrogenase activity, a polypeptide having 3-oxoacyl-CoA reductase activity, an polypeptide having enoyl-CoA hydratase, a polypeptide having trans-2-enoyl-CoA reductase, and a polypeptide having thioesterase activity, or using recombinant host cells expressing one or more of such enzymes. This invention also relates to methods for converting 6-hydroxyhexanoic acid to one or more of adipic acid, 6-aminohexanoic acid, hexamethylenediamine, caprolactam, and 1,6-hexanediol using one or more isolated enzymes such as a polypeptide having dehydrogenase activity, a polypeptide having reductase activity, a polypeptide having hydratase activity, a polypeptide having thioesterase activity, a polypeptide having monooxygenase activity, a polypeptide having transaminase activity or using recombinant host cells expressing one or more such enzymes.

### BACKGROUND

Nylons are polyamides that are generally synthesized by the condensation polymerization of a diamine with a dicarboxylic acid. Similarly, Nylons also may be produced by the condensation polymerization of lactams. A ubiquitous nylon is Nylon 6,6, which is produced by reaction of hexamethylenediamine (HMD) and adipic acid. Nylon 6 can be produced by a ring opening polymerization of caprolactam. Therefore, adipic acid, hexamethylenediamine and caprolactam are important intermediates in the production of Nylons (Anton & Baird, Polyamides Fibers, Encyclopedia of Polymer Science and Technology, 2001).

Industrially, adipic acid and caprolactam are produced via air oxidation of cyclohexane. The air oxidation of cyclohexane produces, in a series of steps, a mixture of cyclohexanone (K) and cyclohexanol (A), designated as KA oil. Nitric acid oxidation of KA oil produces adipic acid (Musser, Adipic acid, Ullmann's Encyclopedia of Industrial Chemistry, 2000). Caprolactam is produced from cyclohexanone via its oxime and subsequent acid rearrangement (Fuchs, Kieczka and Moran, Caprolactam, Ullmann's Encyclopedia of Industrial Chemistry, 2000)

Industrially, hexamethylenediamine (HMD) is produced by hydrocyanation of C6 building block to adiponitrile, followed by hydrogenation to HMD (Herzog and Smiley, Hexamethylenediamine, Ullmann's Encyclopedia of Industrial Chemistry, 2012).

Given a reliance on petrochemical feedstocks; biotechnology offers an alternative approach via biocatalysis. Biocatalysis is the use of biological catalysts, such as enzymes, to perform biochemical transformations of organic compounds.

Both bioderived feedstocks and petrochemical feedstocks are viable starting materials for the biocatalysis processes.

### SUMMARY

Accordingly, against this background, it is clear that there is a need for sustainable methods for producing one or more of adipic acid, caprolactam, 6-aminohexanoic acid, 6-hydroxyhexanoic acid, hexamethylenediamine, and 1,6-hexanediol, where the methods are biocatalyst based.

This document is based at least in part on the discovery that it is possible to construct biochemical pathways for using, inter alia, a β-ketothiolase to produce 6-hydroxyhexanoate, which can be converted in one or more enzymatic steps to adipic acid, 6-aminohexanoic acid, hexamethylenediamine, caprolactam, or 1,6-hexanediol. Adipic acid and adipate, 6-hydroxyhexanoic acid and 6-hydroxyhexanoate, and 6-aminohexanoic and 6-aminohexanoate are used interchangeably herein to refer to the compound in any of its neutral or ionized forms, including any salt forms thereof. It is understood by those skilled in the art that the specific form will depend on pH.

In the face of the optimality principle, it surprisingly has been discovered that appropriate non-natural pathways, feedstocks, host microorganisms, attenuation strategies to the host's biochemical network, and cultivation strategies may be combined to efficiently produce 6-hydroxyhexanoate as a C6 building block, or convert 6-hydroxyhexanoate to other C6 building blocks such as adipic acid, 6-aminohexanoic acid, hexamethylenediamine, caprolactam, or 1,6-hexanediol.

In some embodiments, a terminal carboxyl group can be enzymatically formed using a thioesterase, an aldehyde dehydrogenase, a 6-oxohexanoate dehydrogenase, a 7-oxoheptanoate dehydrogenase, or a monooxgenase (e.g., in combination with an oxidoreductase and ferredoxin). See FIG. 1 and FIG. 2.

In some embodiments, a terminal amine group can be enzymatically formed using a ω-transaminase or a deacylase. See FIG. 4. The ω-transaminase can have at least 70% sequence identity to any one of the amino acid sequences set forth in SEQ ID NOs. 7-12.

In some embodiments, a terminal hydroxyl group can be enzymatically formed using an alcohol dehydrogenase. See FIG. 1 and FIG. 5.

In one aspect, this document features a method of producing 3-oxo-6-hydroxyhexanoyl-CoA. The method includes enzymatically converting 4-hydroxybutyryl-CoA to 3-oxo-6-hydroxyhexanoyl-CoA using a polypeptide having β-ketothiolase activity classified under EC. 2.3.1.- (e.g., EC 2.3.1.16 or EC 2.3.1.174). The polypeptide having β-ketothiolase activity can have at least 70% sequence identity to the amino acid sequence set forth in SEQ ID NO:1, SEQ ID NO:13 or SEQ ID NO: 14. The method can include enzymatically converting 3-oxo-6-hydroxyhexanoyl-CoA to 6-hydroxyhexanoate using a 3-hydroxyacyl-CoA dehydrogenase or a 3-oxoacyl-CoA reductase, an enoyl-CoA hydratase, a trans-2-enoyl-CoA reductase, and a thioesterase or a CoA transferase. The 3-hydroxyacyl-CoA dehydrogenase or 3-oxoacyl-CoA reductase can be classified under EC 1.1.1.35, EC 1.1.1.36, EC 1.1.1.100, or EC 1.1.1.157. The enoyl-CoA hydratase can be classified under EC 4.2.1.17 or EC 4.2.1.119. The trans-2-enoyl-CoA reductase can be classified under EC 1.3.1.38, EC 1.3.1.44, or EC 1.3.1.8. The trans-enoyl-CoA reductase can have at least 70% sequence identity to the amino acid sequence set forth in SEQ ID NO: 15 or SEQ ID NO: 16.

In one aspect, this document features a method for biosynthesizing 6-hydroxyhexanoate. The method includes enzymatically synthesizing 3-oxo-6-hydroxyhexanoyl-CoA from 4-hydroxybutyryl-CoA using a β-ketothiolase classified under EC. 2.3.1.- (e.g., EC 2.3.1.16 or EC 2.3.1.174) and enzymatically converting 3-oxo-6-hydroxyhexanoyl-CoA to 6-hydroxyhexanoate. The β-ketothiolase can have at least 70% sequence identity to the amino acid sequence set forth in SEQ ID NO:1, SEQ ID NO:13 or SEQ ID NO: 14. 3-oxo-6-hydroxyhexanoyl-CoA can be converted to 3-hydroxy-6-hydroxyhexanoyl-CoA using a 3-hydroxyacyl-CoA dehydrogenase or a 3-oxoacyl-CoA reductase, 3-hydroxy-6-hydroxyhexanoyl-CoA can be converted to 2,3-dehydro-6-hydroxyhexanoyl-CoA using an enoyl-CoA hydratase, 2,3-dehydro-6-hydroxyhexanoyl-CoA can be converted to 6-hydroxyhexanoyl-CoA using a trans-2-enoyl-CoA reductase, and 6-hydroxyhexanoyl-CoA can be converted to 6-hydroxyhexanoate using a thioesterase or a CoA transferase.

Any of the methods further can include enzymatically converting 6-hydroxyhexanoate to adipic acid, 6-aminohexanoate, caprolactam, hexamethylenediamine, or 1,6-hexanediol in one or more steps.

For example, 6-hydroxyhexanoate can be enzymatically converted to adipic acid using one or more of a monooxygenase, an alcohol dehydrogenase, a 4-hydroxybutanoate dehydrogenase, a 5-hydroxyvalerate dehydrogenase, a 6-hydroxyhexanoate dehydrogenase, a 7-oxoheptanoate dehydrogenase, a 6-oxohexanoate dehydrogenase, a 5-oxovalerate dehydrogenase, or an aldehyde dehydrogenase.

For example, 6-hydroxyhexanoate can be converted to 6-aminohexanoate using one or more of an alcohol dehydrogenase, a 6-hydroxyhexanoate dehydrogenase, a 5-hydroxypentanoate dehydrogenase, a 4-hydroxybutyrate dehydrogenase, and a ω-transaminase. The ω-transaminase can have at least 70% sequence identity to any one of the amino acid sequences set forth in SEQ ID NO. 7-12.

For example, 6-hydroxyhexanoate can be converted to caprolactam using one or more of an alcohol dehydrogenase, a 6-hydroxyhexanoate dehydrogenase, a 5-hydroxypentanoate dehydrogenase, a 4-hydroxybutyrate dehydrogenase, a ω-transaminase, and an amidohydrolase. The ω-transaminase can have at least 70% sequence identity to any one of the amino acid sequences set forth in SEQ ID NO. 7-12.

For example, 6-hydroxyhexanoate can be converted to hexamethylenediamine using one or more of a carboxylate reductase, a ω-transaminase, an alcohol dehydrogenase, an N-acetyltransferase, and an acetylputrescine deacylase. The ω-transaminase can have at least 70% sequence identity to any one of the amino acid sequences set forth in SEQ ID NO. 7-12.

For example, 6-hydroxyhexanoate can be converted to 1,6-hexanediol using a carboxylate reductase and an alcohol dehydrogenase. The carboxylate reductase can have at least 70% sequence identity to any one of the amino acid sequences set forth in SEQ D NO. 2-6.

In any of the methods, 4-hydroxybutyryl-CoA can be enzymatically produced from 2-oxoglutarate. For example, 4-hydroxybutyryl-CoAcan be enzymatically produced from 2-oxoglutarate using one or more of a glutamate synthase; a 2-oxoglutarate decarboxylase; a branch chain decarboxylase; a glutamate decarboxylase; a ω-transaminase; a CoA transferase, a CoA ligase, and an alcohol dehydrogenase.

In any of the methods described herein, adipic acid can be produced by forming the second terminal functional group in adipate semialdehyde (also known as 6-oxohexanoate) using (i) an aldehyde dehydrogenase classified under EC 1.2.1.3, (ii) a 6-oxohexanoate dehydrogenase classified under EC 1.2.1.63 such as that encoded by ChnE or a 7-oxoheptanoate dehydrogenase classified under EC 1.2.1.- (e.g., the gene product of ThnG) or iii) a monooxgenase in the cytochrome P450 family.

In any of the methods described herein, 6-aminohexanoic acid can be produced by forming the second terminal functional group in adipate semialdehyde using a ω-transaminase classified under EC 2.61.18, EC 2.6.1.19, EC 2.6.1.29, EC 2.6.1.48, or EC 2.6.1.82.

In any of the methods described herein, caprolactam can be produced from 6-aminohexanoic acid using an amidohydrolase classified under EC 3.5.2.-. The amide bond associated with caprolactam is produced from a terminal carboxyl group and terminal amine group of 6-aminohexanoate.

In any of the methods described herein, hexamethylenediamine can be produced by forming a second terminal functional group in (i) 6-aminohexanal using a ω-transaminase classified under EC 2.61.18, EC 2.6.1.19, EC 2.6.1.29, EC 2.6.1.48 or EC 2.6.1.82 or in (ii) N6-acetyl-1,6-diaminohexane using a deacylase classified, for example, under EC 3.5.1.17.

In any of the methods described herein, 1,6 hexanediol can be produced by forming the second terminal functional group in 6-hydroxyhexanal using an alcohol dehydrogenase classified under EC 1.1.1.- (e.g., EC 1.1.1.1, 1.1.1.2, 1.1.1.21, or 1.1.1.184) such as that encoded by YMR318C, YqhD or CAA81612.1.

In some embodiments, the biological feedstock can be or can derive from, monosaccharides, disaccharides, lignocellulose, hemicellulose, cellulose, lignin, levulinic acid and formic acid, triglycerides, glycerol, fatty acids, agricultural waste, condensed distillers' solubles, or municipal waste.

In some embodiments, the non-biological feedstock can be or can derive from natural gas, syngas, CO₂/H₂, methanol, ethanol, benzoate, non-volatile residue (NVR) or a caustic wash waste stream from cyclohexane oxidation processes, or terephthalic acid/isophthalic acid mixture waste streams.

In some embodiments, the host microorganism's tolerance to high concentrations of one or more C6 building blocks is improved through continuous cultivation in a selective environment.

In some embodiments, the host microorganism's biochemical network is attenuated or augmented to (1) ensure the intracellular availability of acetyl-CoA and 4-hydroxybutyryl-CoA, (2) create an NADH or NADPH imbalance that may only be balanced via the formation of one or more C6 building blocks, (3) prevent degradation of central metabolites, central precursors leading to and including C6 building blocks and (4) ensure efficient efflux from the cell.

In some embodiments, a cultivation strategy is used to achieve anaerobic, micro-aerobic, or aerobic cultivation conditions.

In some embodiments, the cultivation strategy includes limiting nutrients, such as limiting nitrogen, phosphate or oxygen.

In some embodiments, one or more C6 building blocks are produced by a single type of microorganism, e.g., a recombinant host containing one or more exogenous nucleic acids, using, for example, a fermentation strategy.

In another aspect, this document features a recombinant host that includes at least one exogenous nucleic acid encoding (i) a β-ketothiolase, (ii) a thioesterase or a CoA transferase, and one or more of (iii) a 3-hydroxyacyl-CoA dehydrogenase or a 3-oxoacyl-CoA reductase, (iv) an enoyl-CoA hydratase, and (v) a trans-2-enoyl-CoA reductase, the host producing 6-hydroxyhexanoate.

A host producing 6-hydroxyhexanoate further can include one or more of the following exogenous enzymes: a monooxygenase, an alcohol dehydrogenase, a 4-hydroxybutanoate dehydrogenase, a 5-hydroxyvalerate dehydrogenase, a 6-hydroxyhexanoate dehydrogenase, a 7-oxoheptanoate dehydrogenase, a 6-oxohexanoate dehydrogenase, a 5-oxovalerate dehydrogenase, or an aldehyde dehydrogenase, the host further producing adipic acid.

A host producing 6-hydroxyhexanoate further can include one or more of the following exogenous enzymes: a monooxygenase, a transaminase, a 6-hydroxyhexanoate dehydrogenase, a 5-hydroxypentanoate dehydrogenase, a 4-hydroxybutyrate dehydrogenase, and an alcohol dehydrogenase, the host further producing 6-aminohexanoate. Such a host further can include an exogenous amidohydrolase, the host further producing caprolactam.

A host producing 6-hydroxyhexanoate further can include one or more of the following exogenous enzymes: a carboxylate reductase, a ω-transaminase, a deacylase, a N-acetyl transferase, or an alcohol dehydrogenase, said host further producing hexamethylenediamine.

A host producing 6-hydroxyhexanoate further can include an exogenous carboxylate reductase and an exogenous alcohol dehydrogenase, the host further producing 1,6-hexanediol.

Any of the recombinant hosts described herein further can include one or more of the following exogenous enzymes: a glutamate synthase; a 2-oxoglutarate decarboxylase; a branch-chain decarboxylase; a glutamate decarboxylase; a ω-transaminase; a CoA-ligase; a CoA-transferase; and an alcohol dehydrogenase.

Any of the recombinant hosts can be a prokaryote such as a prokaryote from a genus selected from the group consisting of Escherichia; Clostridia; Corynebacteria; Cupriavidus; Pseudomonas; Delftia; Bacilluss; Lactobacillus; Lactococcus; and Rhodococcus. For example, the prokaryote can be selected from the group consisting of Escherichia coli, Clostridium ljungdahlii, Clostridium autoethanogenum, Clostridium kluyveri, Corynebacterium glutamicum, Cupriavidus necator, Cupriavidus metallidurans. Pseudomonas fluorescens, Pseudomonas putida, Pseudomonas oleavorans, Delftia acidovorans, Bacillus subtillis, Lactobacillus delbrueckii, Lactococcus lactis, and Rhodococcus equi. Such prokaryotes also can be sources of genes for constructing recombinant host cells described herein that are capable of producing C6 building blocks. Any of the recombinant hosts can be a eukaryote such as a eukaryote from a genus selected from the group consisting of Aspergillus, Saccharomyces, Pichia, Yarrowia, Issatchenkia, Debaryomyces, Arxula, and Kluyveromyces. For example, the eukaryote can be selected from the group consisting of Aspergillus niger, Saccharomyces cerevisiae, Pichia pastoris, Yarrowia lipolytica, Issathenkia orientalis, Debaryomyces hansenii, Arxula adenoinivorans, and Kluyveromyces lactis. Such eukaryotes also can be sources of genes for constructing recombinant host cells described herein that are capable of producing C6 building blocks.

Any of the recombinant hosts described herein further can include attenuation of one or more of the following enzymes: a polyhydroxyalkanoate synthase, an acetyl-CoA thioesterase, a phosphotransacetylase forming acetate, an acetate kinase, a lactate dehydrogenase, a menaquinol-fumarate oxidoreductase, an alcohol dehydrogenase forming ethanol, a triose phosphate isomerase, a pyruvate decarboxylase, a glucose-6-phosphate isomerase, NADH-consuming transhydrogenase, an NADH-specific glutamate dehydrogenase, a NADH/NADPH-utilizing glutamate dehydrogenase, a pimeloyl-CoA dehydrogenase; an acyl-CoA dehydrogenase accepting C6 building blocks and central precursors as substrates; a butyryl-CoA dehydrogenase; or an adipyl-CoA synthetase.

Any of the recombinant hosts described herein further can overexpress one or more genes encoding: an acetyl-CoA synthetase, a 6-phosphogluconate dehydrogenase; a transketolase; a puridine nucleotide transhydrogenase; a glyceraldehyde-3P-dehydrogenase; a malic enzyme; a glucose-6-phosphate dehydrogenase; a glucose dehydrogenase; a fructose 1,6 diphosphatase; a L-alanine dehydrogenase; a L-glutamate dehydrogenase; a formate dehydrogenase; a L-glutamine synthetase; a diamine transporter; a dicarboxylate transporter; and/or a multidrug transporter.

Many of the enzymes described herein catalyze reversible reactions, and the reaction of interest may be the reverse of the described reaction. The schematic pathways shown in FIGS. 1 to 5 illustrate the reaction of interest for each of the intermediates.

Is some aspects, this document features nucleic acid constructs and/or expression vectors comprising (a) polynucleotide encoding a polypeptide having β-ketothiolase activity, wherein the polynucleotide is operably linked to one or more heterologous control sequences that direct production of the polypeptide and wherein the polypeptide having β-ketothiolase activity is selected from the group consisting of: (a) a polypeptide having at least 70% sequence identity to the polypeptide of SEQ ID NOs: 1, 13 or 14; (b) a polynucleotide encoding a polypeptide having ω-transaminase activity, wherein the polynucleotide is operably linked to one or more heterologous control sequences that direct production of the polypeptide and wherein the polypeptide having ω-transaminase activity is selected from the group consisting of: (a) a polypeptide having at least 70% sequence identity to the polypeptide of SEQ ID NOs: 7-12; a polynucleotide encoding a polypeptide having carboxylate reductase activity, wherein the polynucleotide is operably linked to one or more heterologous control sequences that direct production of the polypeptide and wherein the polypeptide having carboxylate reductase activity is selected from the group consisting of: (a) a polypeptide having at least 70% sequence identity to the polypeptide of SEQ ID NOs: 2-6; or (d) a polynucleotide encoding a polypeptide having 3-hydroxyacyl-CoA dehydrogenase, 3-oxoacyl-CoA reductase, enoyl-CoA hydratase, trans-2-enoyl-CoA reductase, thioesterase CoA transferase, monooxygenase, an alcohol dehydrogenase, a 4-hydroxybutanoate dehydrogenase, a 5-hydroxyvalerate dehydrogenase, a 6-hydroxyhexanoate dehydrogenase, a 7-oxoheptanoate dehydrogenase, a 6-oxohexanoate dehydrogenase, a 5-oxovalerate dehydrogenase, aldehyde dehydrogenase, a 6-hydroxyhexanoate dehydrogenase, a 5-hydroxypentanoate dehydrogenase, a ω-transaminase, amidohydrolase, glutamate synthase; a 2-oxoglutarate decarboxylase; a branch chain decarboxylase; a glutamate decarboxylase; a ω-transaminase; a CoA transferase, a CoA ligase activity. In some embodiments, the disclosure provides composition comprising the nucleic acid construct or expression vector described above.

In one aspect, this document features a method for producing a bioderived six carbon compound. The method for producing a bioderived six carbon compound can include culturing or growing a recombinant host as described herein under conditions and for a sufficient period of time to produce the bioderived six carbon compound, wherein, optionally, the bioderived six carbon compound is selected from the group consisting of adipic acid, 6-aminohexanoic acid, hexamethylenediamine, caprolactam, or 1,6-hexanediol, and combinations thereof.

In one aspect, this document features composition comprising a bioderived six carbon compound as described herein and a compound other than the bioderived six carbon compound, wherein the bioderived six carbon compound is selected from the group consisting of adipic acid, 6-aminohexanoic acid, hexamethylenediamine, caprolactam, or 1,6-hexanediol, and combinations thereof. For example, the bioderived six carbon compound is a cellular portion of a host cell or an organism.

This document also features a biobased polymer comprising the bioderived adipic acid, 6-aminohexanoic acid, hexamethylenediamine, caprolactam, or 1,6-hexanediol, and combinations thereof.

This document also features a biobased resin comprising the bioderived adipic acid, 6-aminohexanoic acid, hexamethylenediamine, caprolactam, or 1,6-hexanediol, and combinations thereof, as well as a molded product obtained by molding a biobased resin.

In another aspect, this document features a process for producing a biobased polymer that includes chemically reacting the bioderived adipic acid, 6-aminohexanoic acid, hexamethylenediamine, caprolactam, or 1,6-hexanediol, with itself or another compound in a polymer producing reaction.

In another aspect, this document features a process for producing a biobased resin that includes chemically reacting the bioderived adipic acid, 6-aminohexanoic acid, hexamethylenediamine, caprolactam, or 1,6-hexanediol, with itself or another compound in a resin producing reaction.

Also, described herein is a biochemical network comprising a polypeptide having β-ketothiolase activity, wherein the polypeptide having β-ketothiolase activity enzymatically converts 4-hydroxybutyryl-CoA to 3-oxo-6-hydroxyhexanoyl-CoA.

The biochemical network can further include a polypeptide having 3-hydroxyacyl-CoA dehydrogenase activity or a polypeptide having 3-oxoacyl-CoA reductase activity, a polypeptide having enoyl-CoA hydratase activity, a polypeptide having trans-2-enoyl-CoA reductase activity, and a polypeptide having thioesterase or a CoA transferase activity for enzymatically converting 3-oxo-6-hydroxyhexanoyl-CoA to 6-hydroxyhexanoate.

The biochemical network can further include one or more polypeptides having monooxygenase, alcohol dehydrogenase, 4-hydroxybutanoate dehydrogenase, 5-hydroxyvalerate dehydrogenase, 6-hydroxyhexanoate dehydrogenase, 7-oxoheptanoate dehydrogenase, 6-oxohexanoate dehydrogenase, 5-oxovalerate dehydrogenase, or aldehyde dehydrogenase activity for enzymatically converting 6-hydroxyhexanoate to adipic acid.

The biochemical network can further include a polypeptide having ω-transaminase activity for enzymatically converting 6-hydroxyhexanoate to 6-aminohexanoic acid.

The biochemical network can further include a polypeptide having amidohydrolase activity for enzymatically converting 6-aminohexanoic acid to caprolactam.

The biochemical network can further include one or more polypeptides having a ω-transaminase or deacylase activity for enzymatically converting 6-hydroxyhexanoate to hexamethylenediamine.

The biochemical network can further include one or more polypeptides having alcohol dehydrogenase activity 1,6 hexanediol by forming the second terminal functional group in 6-hydroxyhexanal.

In one aspect, the biochemical network is a non-naturally occurring biochemical network comprising at least one substrate of FIG. 1 to FIG. 5, at least one exogenous nucleic acid encoding a polypeptide having the activity of at least one enzyme of FIG. 1 to FIG. 5 and at least one product of FIG. 1 to FIG. 5.

In one aspect of the invention, described is a step for forming at least one compound of FIG. 1 to FIG. 5. In one aspect of the invention, described is a means for forming at least one compound of FIG. 1 to FIG. 5. Also, described herein is a means for obtaining adipic acid, 6-aminohexanoic acid, hexamethylenediamine, caprolactam, or 1,6-hexanediol using one or more polypeptides having β-ketothiolase, 3-hydroxyacyl-CoA dehydrogenase, 3-oxoacyl-CoA reductase, enoyl-CoA hydratase, trans-2-enoyl-CoA reductase, thioesterase or a CoA transferase, monooxygenase, alcohol dehydrogenase, 4-hydroxybutanoate dehydrogenase, 5-hydroxyvalerate dehydrogenase, 6-hydroxyhexanoate dehydrogenase, 7-oxoheptanoate dehydrogenase, 6-oxohexanoate dehydrogenase, 5-oxovalerate dehydrogenase, aldehyde dehydrogenase, ω-transaminase, amidohydrolase, ω-transaminase or deacylase activity.

In another aspect, this document features a composition comprising one or more polypeptides having β-ketothiolase, 3-hydroxyacyl-CoA dehydrogenase, 3-oxoacyl-CoA reductase, enoyl-CoA hydratase, trans-2-enoyl-CoA reductase, thioesterase or a CoA transferase, monooxygenase, alcohol dehydrogenase, 4-hydroxybutanoate dehydrogenase, 5-hydroxyvalerate dehydrogenase, 6-hydroxyhexanoate dehydrogenase, 7-oxoheptanoate dehydrogenase, 6-oxohexanoate dehydrogenase, 5-oxovalerate dehydrogenase, aldehyde dehydrogenase, ω-transaminase, amidohydrolase, ω-transaminase or deacylase activity and at least one of adipic acid, 6-aminohexanoic acid, hexamethylenediamine, caprolactam, or 1,6-hexanediol. The composition can be cellular.

One of skill in the art understands that compounds containing carboxylic acid groups (including, but not limited to, organic monoacids, hydroxyacids, aminoacids, and dicarboxylic acids) are formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include, but are not limited to, ethanolamine, diethanolamine, triethanolamine, tromethamine, N-methylglucamine, and the like. Acceptable inorganic bases include, but are not limited to, aluminum hydroxide, calcium hydroxide, potassium hydroxide, sodium carbonate, sodium hydroxide, and the like. A salt of the present invention is isolated as a salt or converted to the free acid by reducing the pH to below the pKa, through addition of acid or treatment with an acidic ion exchange resin.

One of skill in the art understands that compounds containing amine groups (including, but not limited to, organic amines, aminoacids, and diamines) are formed or converted to their ionic salt form, for example, by addition of an acidic proton to the amine to form the ammonium salt, formed with inorganic acids such as hydrochloric acid, hydrobromic acid, sulfuric acid, nitric acid, phosphoric acid, and the like; or formed with organic acids including, but not limited to, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include, but are not limited to, aluminum hydroxide, calcium hydroxide, potassium hydroxide, sodium carbonate, sodium hydroxide, and the like. A salt of the present invention is isolated as a salt or converted to the free amine by raising the pH to above the pKb through addition of base or treatment with a basic ion exchange resin.

One of skill in the art understands that compounds containing both amine groups and carboxylic acid groups (including, but not limited to, aminoacids) are formed or converted to their ionic salt form by either 1) acid addition salts, formed with inorganic acids including, but not limited to, hydrochloric acid, hydrobromic acid, sulfuric acid, nitric acid, phosphoric acid, and the like; or formed with organic acids including, but not limited to, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include, but are not limited to, aluminum hydroxide, calcium hydroxide, potassium hydroxide, sodium carbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases include, but are not limited to, ethanolamine, diethanolamine, triethanolamine, tromethamine, N-methylglucamine, and the like. Acceptable inorganic bases include, but are not limited to, aluminum hydroxide, calcium hydroxide, potassium hydroxide, sodium carbonate, sodium hydroxide, and the like. A salt can of the present invention is isolated as a salt or converted to the free acid by reducing the pH to below the pKa through addition of acid or treatment with an acidic ion exchange resin.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the application, including the written description and drawings, and the claims. The word “comprising” in the claims may be replaced by “consisting essentially of” or with “consisting of,” according to standard practice in patent law.

### DESCRIPTION OF DRAWINGS

FIG. 1 is a schematic of exemplary biochemical pathways leading to 6-hydroxyhexanoate using 2-oxo-glutarate as a central metabolite.

FIG. 2 is a schematic of exemplary biochemical pathways leading to adipic acid using 6-hydroxyhexanoate as a central precursor.

FIG. 3 is a schematic of an exemplary biochemical pathway leading to 6-aminhexanoate using 6-hydroxyhexanoate as a central precursor and a schematic of an exemplary biochemical pathway leading to caprolactam from 6-aminohexanoate.

FIG. 4 is a schematic of exemplary biochemical pathways leading to hexamethylenediamine using 6-aminohexanoate, 6-hydroxyhexanoate, adipate semialdehyde, or 1,6-hexanediol as a central precursor.

FIG. 5 is a schematic of an exemplary biochemical pathway leading to 1,6-hexanediol using 6-hydroxyhexanoate as a central precursor.

FIG. 6 contains the amino acid sequences of a Cupriavidus necator β-ketothiolase (see GenBank Accession No. AAC38322.1, SEQ ID NO: 1), a Mycobacterium marinum carboxylate reductase (see Genbank Accession No. ACC40567.1, SEQ ID NO: 2), a Mycobacterium smegmatis carboxylate reductase (see Genbank Accession No. ABK71854.1, SEQ ID NO: 3), a Segniliparus rugosus carboxylate reductase (see Genbank Accession No. EFV11917.1, SEQ ID NO: 4), a Mycobacterium massiliense carboxylate reductase (see Genbank Accession No. EIV11143.1, SEQ ID NO: 5), a Segniliparus rotundus carboxylate reductase (see Genbank Accession No. ADG98140.1, SEQ ID NO: 6), a Chromobacterium violaceum ω-transaminase (see Genbank Accession No. AAQ59697.1, SEQ ID NO: 7), a Pseudomonas aeruginosa ω-transaminase (see Genbank Accession No. AAG08191.1, SEQ ID NO: 8), a Pseudomonas syringae CD-transaminase (see Genbank Accession No. AAY39893.1, SEQ ID NO: 9), a Rhodobacter sphaeroides ω-transaminase (see Genbank Accession No. ABA81135.1, SEQ ID NO: 10), an Escherichia coli ω-transaminase (see Genbank Accession No. AAA57874.1, SEQ ID NO: 11), a Vibrio fluvialis ω-transaminase (See Genbank Accession No. AEA39183.1, SEQ ID NO: 12), an Escherichia coli β-ketothiolase (see GenBank Accession No. AAC74479.1, SEQ ID NO: 13), a Clostridium aminobutyricum CoA-transferase (see GenBank Accession No. CAB60036.2, SEQ ID NO: 14), a Treponema denticola enoyl-CoA reductase (see GenBank Accession No. AAS11092.1, SEQ ID NO: 15), an Euglena gracilis enoyl-CoA reductase (see GenBank Accession No. AAW66853.1, SEQ ID NO: 16) and a Salmonella typhimurium decarboxylase (see GenBank Accession No. CAC48239.1, SEQ ID NO: 17).

FIG. 7 is a bar graph summarizing the change in absorbance at 340 nm after 20 minutes, which is a measure of the consumption of NADPH and the activity of the carboxylate reductases of the enzyme only controls (no substrate).

FIG. 8 is a bar graph of the change in absorbance at 340 nm after 20 minutes, which is a measure of the consumption of NADPH and the activity of carboxylate reductases for converting 6-hydroxyhexanoate to 6-hydroxyhexanal relative to the empty vector control.

FIG. 9 is a bar graph of the change in absorbance at 340 nm after 20 minutes, which is a measure of the consumption of NADPH and the activity of carboxylate reductases for converting N6-acetyl-6-aminohexanoate to N6-acetyl-6-aminohexanal relative to the empty vector control.

FIG. 10 is a bar graph of the change in absorbance at 340 nm after 20 minutes, which is a measure of the consumption of NADPH and activity of carboxylate reductases for converting adipate semialdehyde to hexanedial relative to the empty vector control.

FIG. 11 is a bar graph summarizing the percent conversion after 4 hours of pyruvate to L-alanine (mol/mol) as a measure of the ω-transaminase activity of the enzyme only controls (no substrate).

FIG. 12 is a bar graph of the percent conversion after 24 hours of pyruvate to L-alanine (mol/mol) as a measure of the ω-transaminase activity for converting 6-aminohexanoate to adipate semialdehyde relative to the empty vector control.

FIG. 13 is a bar graph of the percent conversion after 4 hours of L-alanine to pyruvate (mol/mol) as a measure of the ω-transaminase activity for converting adipate semialdehyde to 6-aminohexanoate relative to the empty vector control.

FIG. 14 is a bar graph of the percent conversion after 4 hours of pyruvate to L-alanine (mol/mol) as a measure of the ω-transaminase activity for converting hexamethylenediamine to 6-aminohexanal relative to the empty vector control.

FIG. 15 is a bar graph of the percent conversion after 4 hours of pyruvate to L-alanine (mol/mol) as a measure of the ω-transaminase activity for converting N6-acetyl-1,6-diaminohexane to N6-acetyl-6-aminohexanal relative to the empty vector control.

FIG. 16 is a bar graph of the percent conversion after 4 hours of pyruvate to L-alanine (mol/mol) as a measure of the ω-transaminase activity for converting 6-aminohexanol to 6-oxohexanol relative to the empty vector control.

### DETAILED DESCRIPTION

In general, this document provides enzymes, non-natural pathways, cultivation strategies, feedstocks, host microorganisms and attenuations to the host's biochemical network, for producing 6-hydroxyhexanoate or one or more of adipic acid, caprolactam, 6-aminohexanoic acid, hexamethylenediamine or 1,6-hexanediol, all of which are referred to as C6 building blocks herein. As used herein, the term “central precursor” is used to denote any metabolite in any metabolic pathway shown herein leading to the synthesis of a C6 building block. The term “central metabolite” is used herein to denote a metabolite that is produced in all microorganisms to support growth.

Host microorganisms described herein can include endogenous pathways that can be manipulated such that 6-hydroxyhexanoate or one or more other C6 building blocks can be produced. In an endogenous pathway, the host microorganism naturally expresses all of the enzymes catalyzing the reactions within the pathway. A host microorganism containing an engineered pathway does not naturally express all of the enzymes catalyzing the reactions within the pathway but has been engineered such that all of the enzymes within the pathway are expressed in the host.

The term “exogenous” as used herein with reference to a nucleic acid (or a protein) and a host refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host once in the host. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

For example, depending on the host and the compounds produced by the host, one or more of the following enzymes may be expressed in the host in addition to a (3-ketothiolase: a 3-hydroxyacyl-CoA dehydrogenase, a 3-oxoacyl-CoA reductase, an enoyl-CoA hydratase, a trans-2-enoyl-CoA reductase, a thioesterase, a CoA transferase, an aldehyde dehydrogenase, a monooxygenase, an alcohol dehydrogenase, a 6-oxohexanoate dehydrogenase, a 7-oxoheptanoate dehydrogenase, a co transaminase, a 6-hydroxyhexanoate dehydrogenase, a 5-hydroxypentanoate dehydrogenase, a 4-hydroxybutyrate dehydrogenase, a carboxylate reductase, a deacylase, an N-acetyl transferase, a ω-transaminase, or an amidohydrolase. In recombinant hosts expressing a carboxylate reductase, a phosphopantetheinyl transferase also can be expressed as it enhances activity of the carboxylate reductase. In recombinant hosts expressing a monooxygenase, an electron transfer chain protein such as an oxidoreductase or ferredoxin polypeptide also can be expressed.

For example, a recombinant host can include an exogenous β-ketothiolase and produce 3-oxo-6-hydroxyhexanoyl-CoA, which can be converted to 6-hydroxyhexanoate.

For example, a recombinant host can include an exogenous β-ketothiolase and an exogenous thioesterase or CoA-transferase, and one or more of the following exogenous enzymes: 3-hydroxyacyl-CoA dehydrogenase or a 3-oxoacyl-CoA reductase, an enoyl-CoA hydratase, and a trans-2-enoyl-CoA reductase, and produce 6-hydroxyhexanoate. For example, a recombinant host can include an exogenous β-ketothiolase, an exogenous thioesterase or CoA-transferase, an enoyl-CoA hydratase, an exogenous trans-2-enoyl-CoA reductase, and an exogenous 3-hydroxyacyl-CoA dehydrogenase or an exogenous 3-oxoacyl-CoA reductase, and produce 6-hydroxyhexanoate.

For example, a recombinant host producing 6-hydroxyhexanoate can include one or more of the following exogenous enzymes: a monooxygenase, an alcohol dehydrogenase, a 4-hydroxybutyrate dehydrogenase, a 5-hydroxypentanoate dehydrogenase, a 6-hydroxyhexanoate dehydrogenase, a 7-oxoheptanoate dehydrogenase, a 5-oxovalerate dehydrogenase, a 6-oxohexanoate dehydrogenase, or an aldehyde dehydrogenase, and further produce adipic acid. For example, a recombinant host producing 6-hydroxyhexanoate can include an exogenous monooxygenase and produce adipic acid. For example, a recombinant host producing 6-hydroxyhexanoate can include an exogenous 6-hydroxyhexanoate dehydrogenase and an aldehyde dehydrogenase and produce adipic acid. For example, a recombinant host producing 6-hydroxyhexanoate can include an exogenous alcohol dehydrogenase and one of the following exogenous enzymes: a 5-oxovalerate dehydrogenase, a 6-oxohexanoate dehydrogenase, or a 7-oxoheptanoate dehydrogenase, and produce adipic acid.

For example, a recombinant host producing 6-hydroxyhexanoate can include one or more of the following exogenous enzymes: an alcohol dehydrogenase, a 6-hydroxyhexanoate dehydrogenase, or a transaminase, and further produce 6-aminohexanoate. For example, a recombinant host producing 6-hydroxyhexanoate can include an exogenous alcohol dehydrogenase and an exogenous transaminase and produce 6-aminohexanoate. For example, a recombinant host producing 6-hydroxyhexanoate can include an exogenous 6-hydroxyhexanoate dehydrogenase and an exogenous transaminase and produce 6-aminohexanoate. Any of such hosts further can include an exogenous amidohydrolase and further produce caprolactam.

For example, a recombinant host producing 6-hydroxyhexanoate can include one or more of the following exogenous enzymes: a carboxylate reductase, a ω-transaminase, a deacylase, an N-acetyl transferase, or an alcohol dehydrogenase, and produce hexamethylenediamine. For example, a recombinant host producing 6-hydroxyhexanoate can include an exogenous carboxylate reductase, an exogenous alcohol dehydrogenase, and one or more exogenous transaminases (e.g., one transaminase or two different transaminases), and produce hexamethylenediamine. For example, a recombinant host producing 6-hydroxyhexanoate can include an exogenous carboxylate reductase and one or more exogenous transaminases (e.g., one transaminase or two different transaminases) and produce hexamethylenediamine. For example, a recombinant host producing 6-hydroxyhexanoate can include an exogenous alcohol dehydrogenase, an exogenous carboxylate reductase, and one or more exogenous transaminases (e.g., one transaminase, or two or three different transaminases) and produce hexamethylenediamine. For example, a recombinant host producing 6-hydroxyhexanoate can include an exogenous alcohol dehydrogenase, an exogenous N-acetyl transferase, a carboxylate reductase, a deacylase, and one or more exogenous transaminases (e.g., one transaminase or two different transaminases) and produce hexamethylenediamine.

For example, a recombinant host producing 6-hydroxyhexanoate can include one or more of the following exogenous enzymes: a carboxylate reductase and an exogenous alcohol dehydrogenase, and further produce 1,6-hexanediol.

In any of the recombinant hosts, the recombinant host also can include one or more (e.g., one, two, three, or four) of the following exogenous enzymes used to convert 2-oxoglutrate to 4-hydroxybutyryl-CoA: a glutamate synthase; a 2-oxoglutarate decarboxylase; a branch-chain decarboxylase; a glutamate decarboxylase; a CoA-ligase; a CoA-transferase; a ω-transaminase; and an alcohol dehydrogenase. For example, a recombinant host can include an exogenous glutamate synthase, a glutamate decarboxylase; a CoA-ligase or a CoA-transferase; a ω-transaminase; and an alcohol dehydrogenase. For example, a recombinant host can include an exogenous 2-oxoglutarate decarboxylase or a branch-chain decarboxylase; a CoA-ligase; a CoA-transferase; and an alcohol dehydrogenase.

Within an engineered pathway, the enzymes can be from a single source, i.e., from one species or genera, or can be from multiple sources, i.e., different species or genera. Nucleic acids encoding the enzymes described herein have been identified from various organisms and are readily available in publicly available databases such as GenBank or EMBL.

As used herein, references to a particular enzyme (e.g. (3-ketothiolase) means a polypeptide having the activity of the particular enzyme (e.g. a polypeptide having 0-ketothiolase activity).

Any of the enzymes described herein that can be used for production of one or more C6 building blocks can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of the corresponding wild-type enzyme. It will be appreciated that the sequence identity can be determined on the basis of the mature enzyme (e.g., with any signal sequence removed) or on the basis of the immature enzyme (e.g., with any signal sequence included). It also will be appreciated that the initial methionine residue may or may not be present on any of the enzyme sequences described herein.

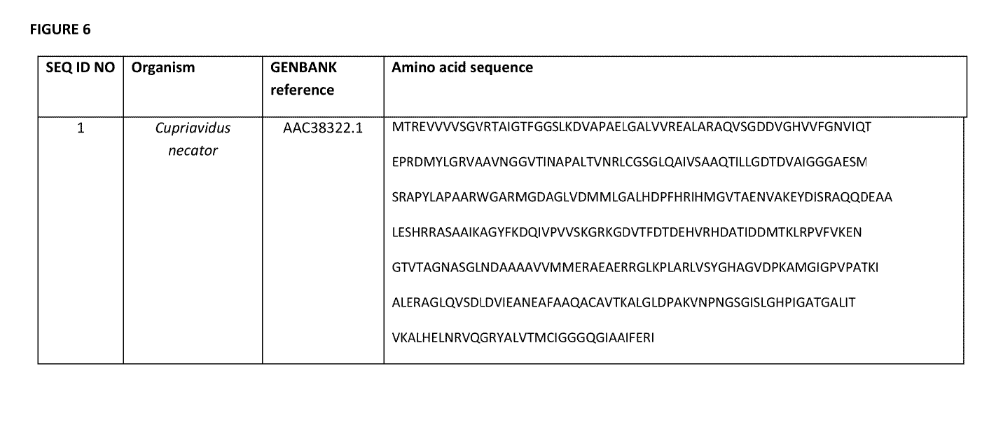

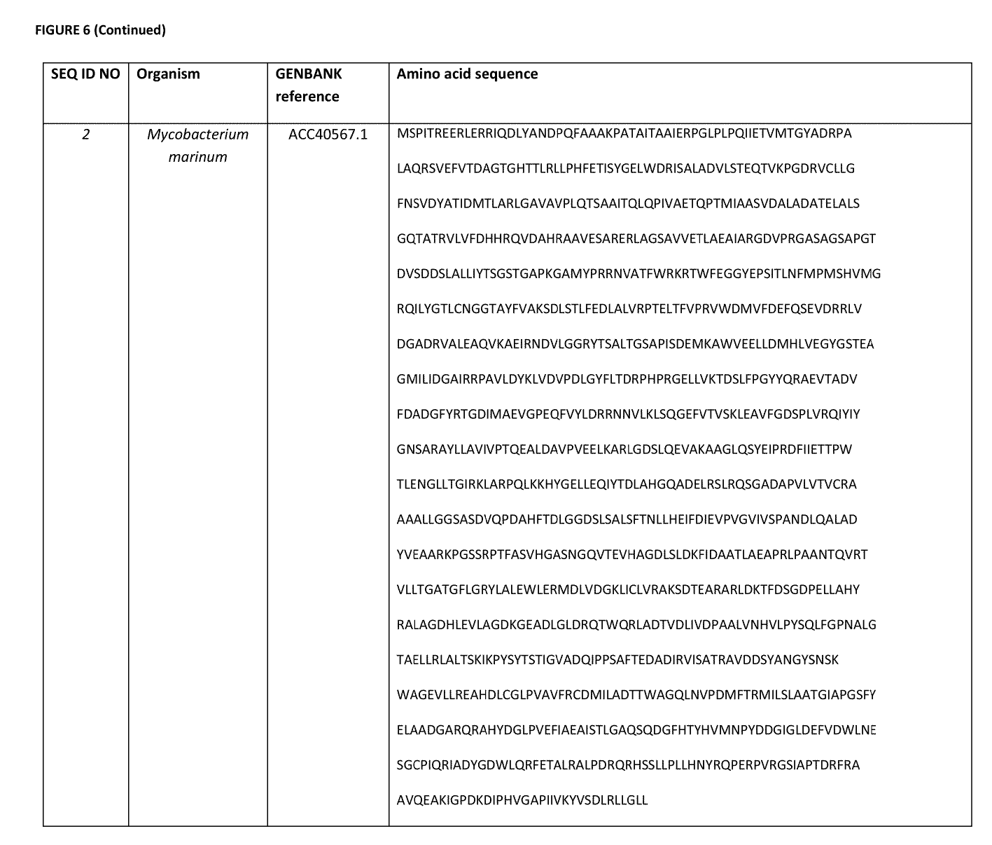

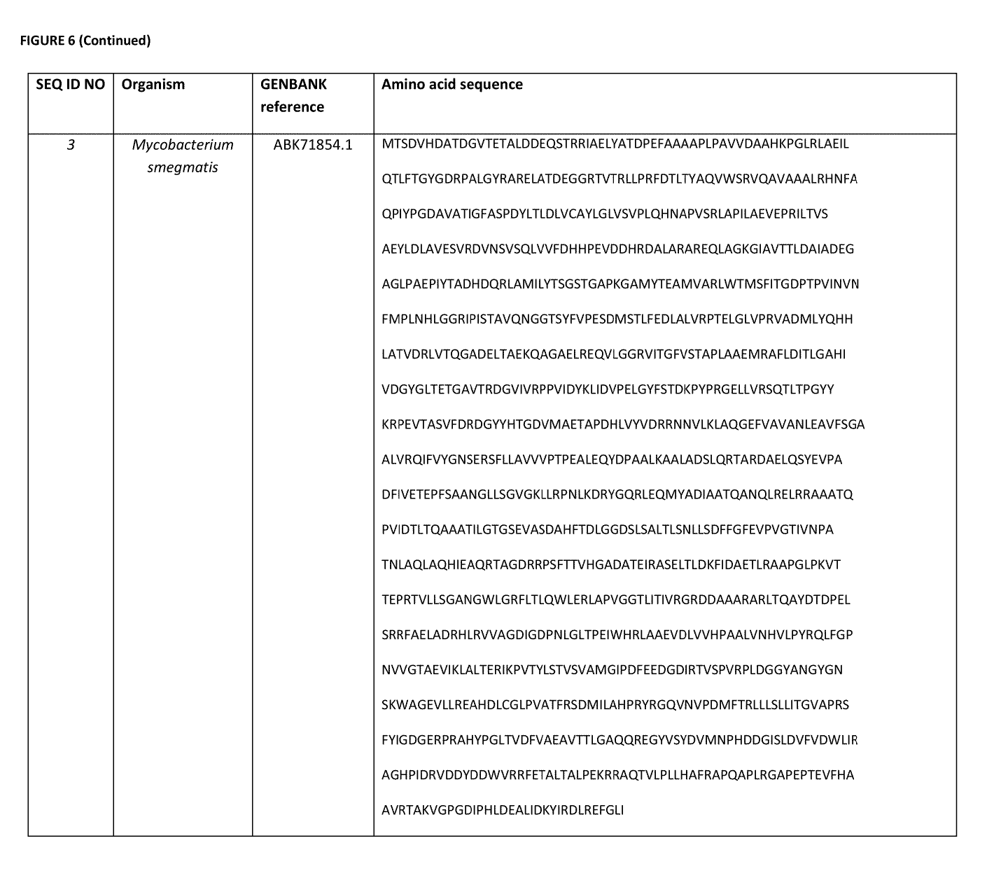

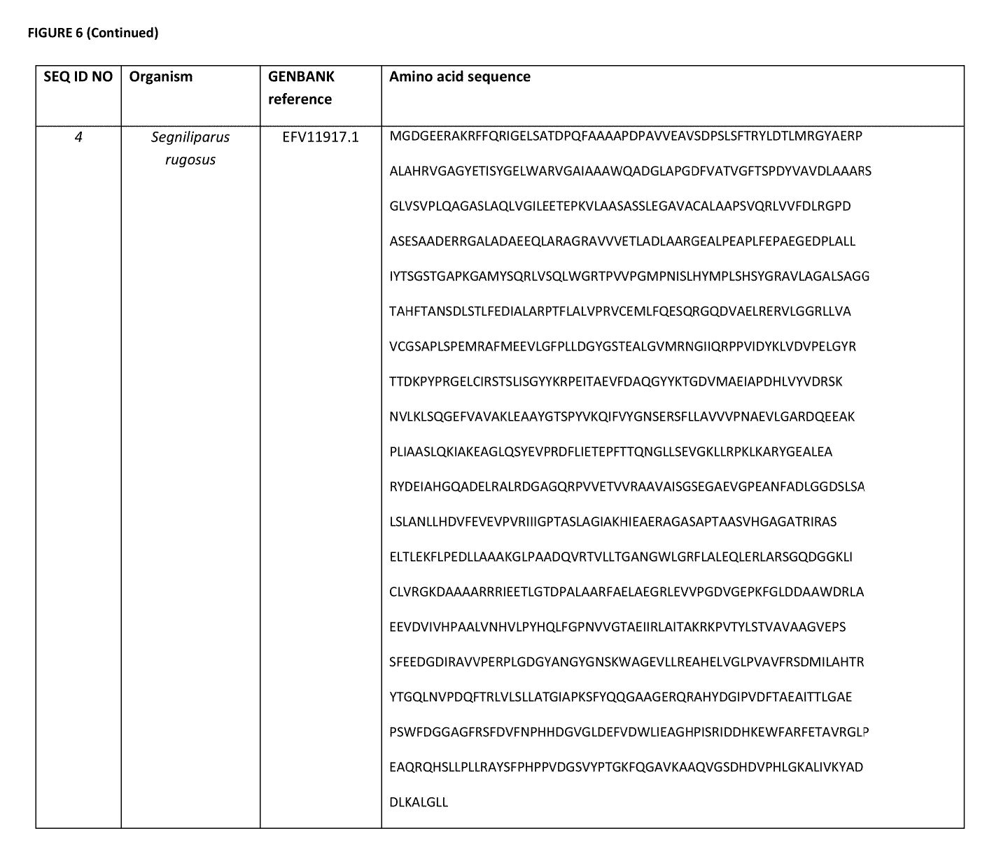

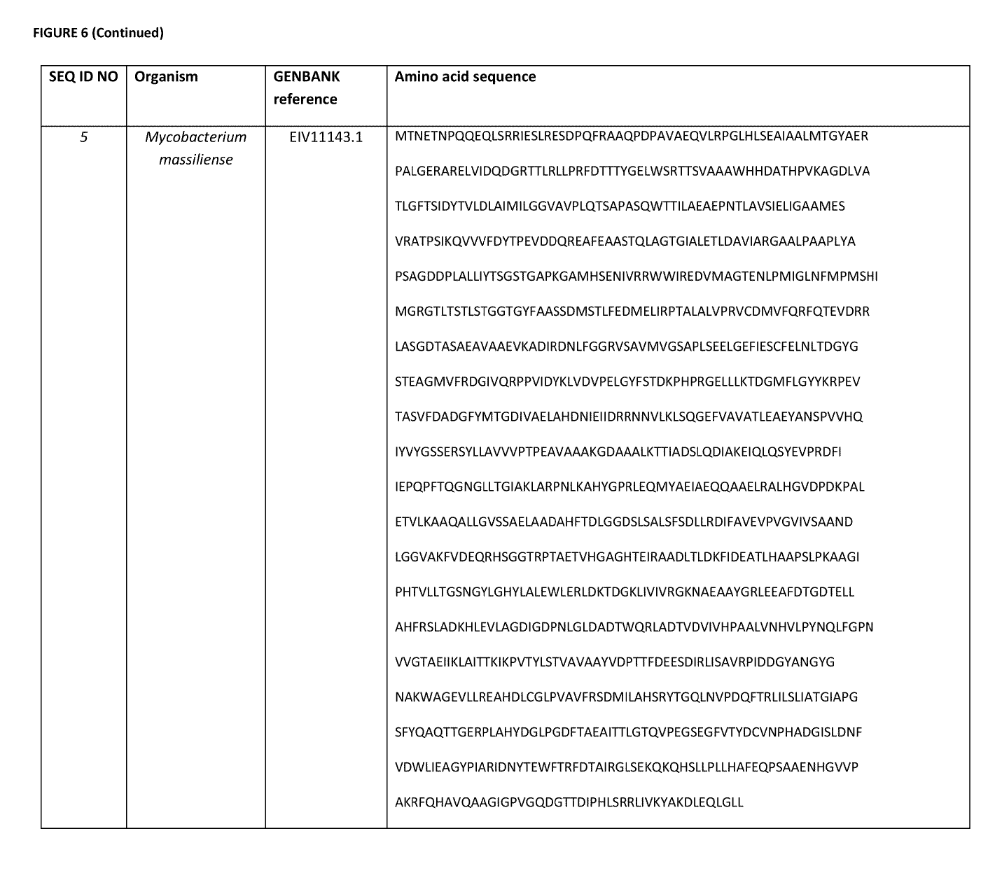

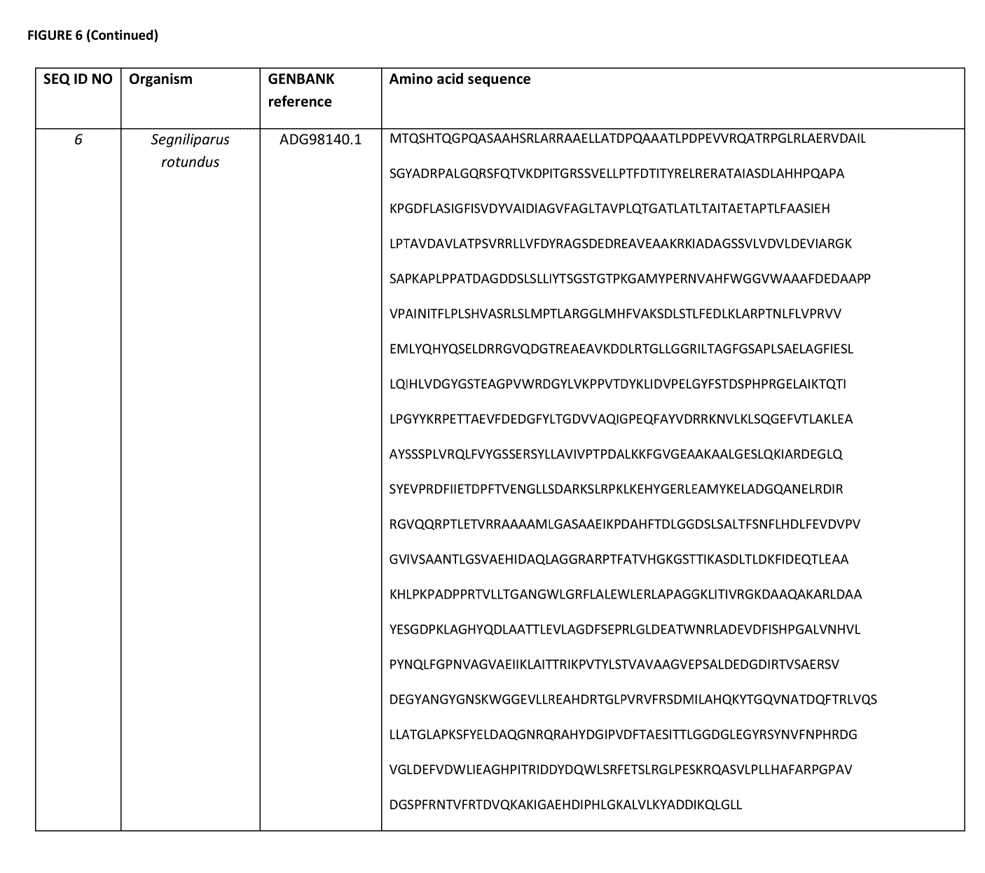

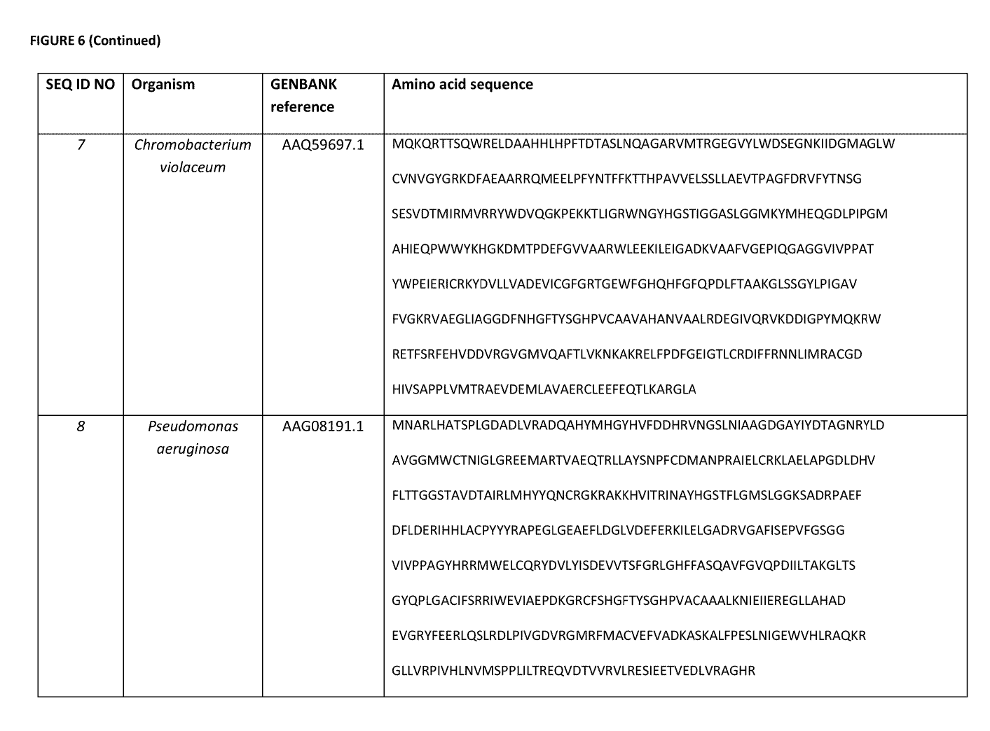

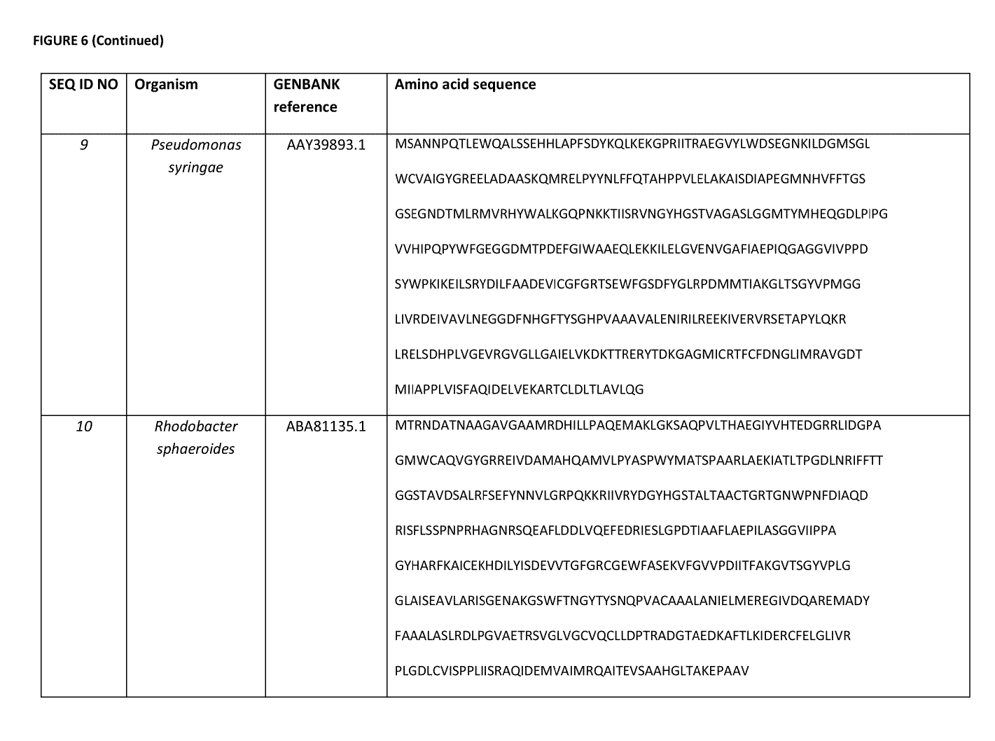

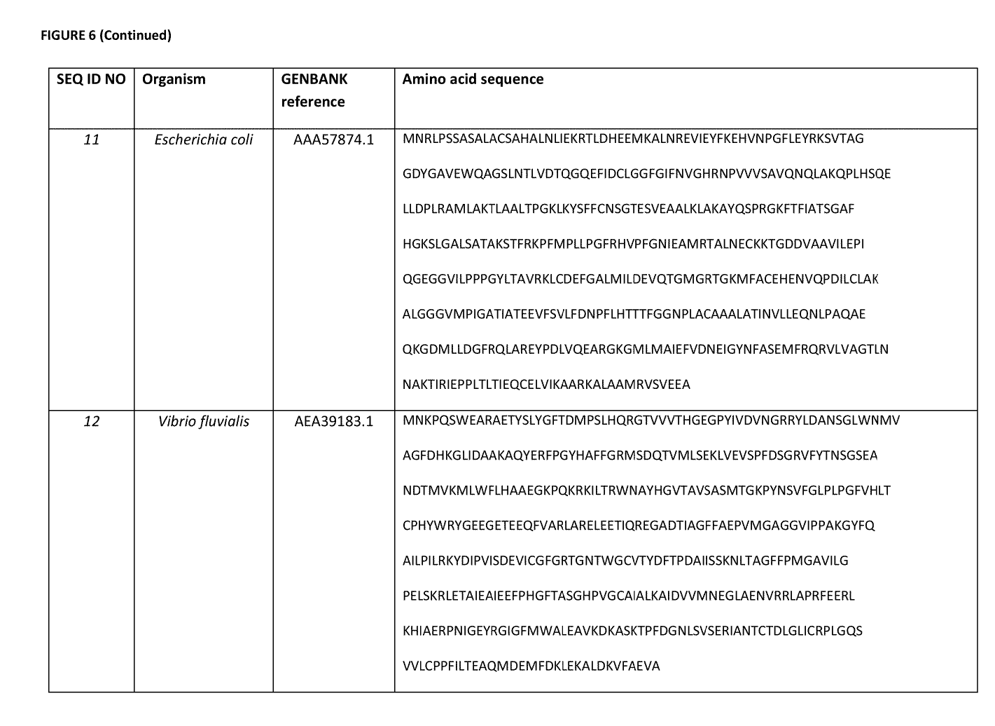

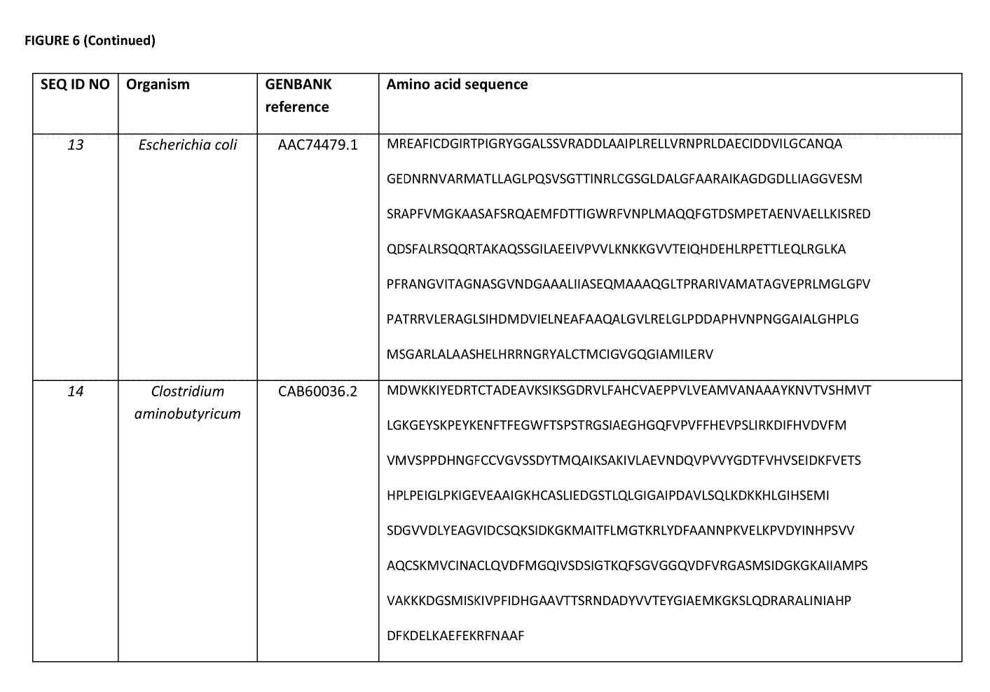

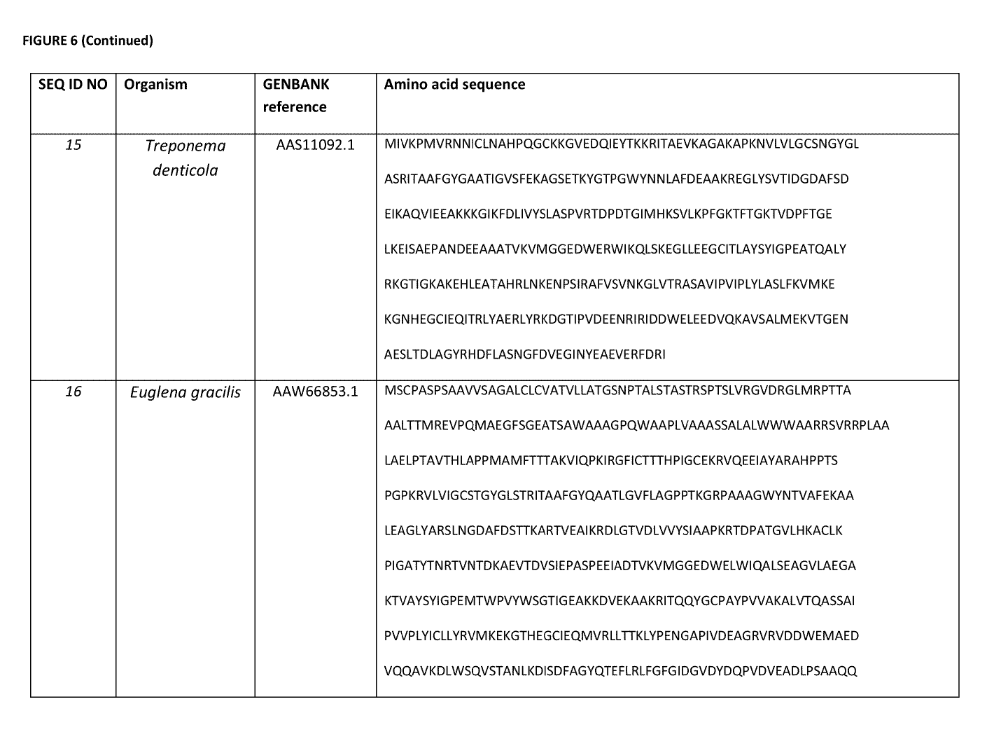

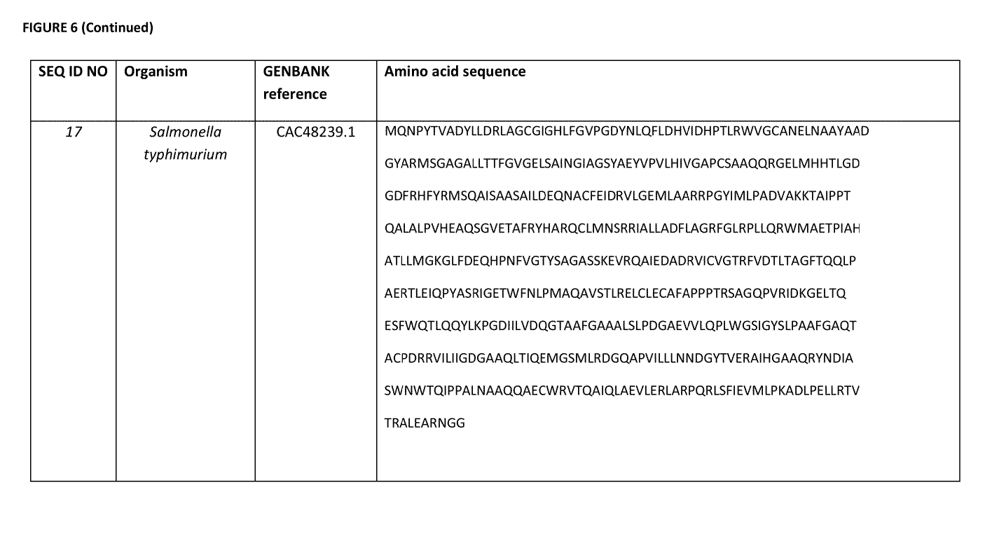

For example, a polypeptide having β-ketothiolase activity described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Cupriavidus necator (see GenBank Accession No. AAC38322.1, SEQ ID NO: 1), an Escherichia coli (see GenBank Accession No. AAC74479.1, SEQ ID NO: 13) β-ketothiolase or a Clostridium aminobutyricum (see GenBank Accession No. CAB60036.2, SEQ ID NO: 14). See FIG. 6.

For example, a carboxylate reductase described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Mycobacterium marinum (see Genbank Accession No. ACC40567.1, SEQ ID NO: 2), a Mycobacterium smegmatis (see Genbank Accession No. ABK71854.1, SEQ ID NO: 3), a Segniliparus rugosus (see Genbank Accession No. EFV11917.1, SEQ ID NO: 4), a Mycobacterium massiliense (see Genbank Accession No. EIV11143.1, SEQ ID NO: 5), or a Segniliparus rotundus (see Genbank Accession No. ADG98140.1, SEQ ID NO: 6) carboxylate reductase. See FIG. 6.

For example, a ω-transaminase described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Chromobacterium violaceum (see Genbank Accession No. AAQ59697.1, SEQ ID NO: 7), a Pseudomonas aeruginosa (see Genbank Accession No. AAG08191.1, SEQ ID NO: 8), a Pseudomonas syringae (see Genbank Accession No. AAY39893.1, SEQ ID NO: 9), a Rhodobacter sphaeroides (see Genbank Accession No. ABA81135.1, SEQ ID NO: 10), an Escherichia coli (see Genbank Accession No. AAA57874.1, SEQ ID NO: 11), or a Vibrio fluvialis (see Genbank Accession No. AEA39183.1, SEQ ID NO: 12) ω-transaminase. Some of these ω-transaminases are diamine ω-transaminases. See, FIG. 6.

For example, an enoyl-CoA reductase described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Treponema denticola (see Genbank Accession No. AAS11092.1, SEQ ID NO: 15) or a Euglena gracilis (see Genbank Accession No. AAW66853.1, SEQ ID NO: 16). See, FIG. 6.

For example, a decarboxylase described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Salmonella typhimurium (see Genbank Accession No. CAC48239.1, SEQ ID NO: 17). See, FIG. 6.

The percent identity (homology) between two amino acid sequences can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., worldwide web address fr.com/blast/) or the U.S. government's National Center for Biotechnology Information web site (worldwide web address ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq i c:\seq1.txt j c:\seq2.txt p blastp o c:\output.txt. If the two compared sequences share homology (identity), then the designated output file will present those regions of homology as aligned sequences. If the two compared sequences do not share homology (identity), then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the enzymes described herein can also be used in the methods of the document. The term “functional fragment” as used herein refers to a peptide fragment of a protein that has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, wild-type protein. The functional fragment can generally, but not always, be comprised of a continuous region of the protein, wherein the region has functional activity.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagglutinin (HA), glutathione-S-transferase (GST), or maltosebinding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Engineered hosts can naturally express none or some (e.g., one or more, two or more, three or more, four or more, five or more, or six or more) of the enzymes of the pathways described herein. Thus, a pathway within an engineered host can include all exogenous enzymes, or can include both endogenous and exogenous enzymes. Endogenous genes of the engineered hosts also can be disrupted to prevent the formation of undesirable metabolites or prevent the loss of intermediates in the pathway through other enzymes acting on such intermediates. Engineered hosts can be referred to as recombinant hosts or recombinant host cells. As described herein recombinant hosts can include nucleic acids encoding one or more of a β-ketothiolase, a dehydrogenase, a synthase, a decarboxylase, a reductase, a hydratase, a thioesterase, a monooxygenase, a thioesterase amidohydrolase, and transaminase as described herein.

In addition, the production of C6 building blocks can be performed in vitro using the isolated enzymes described herein, using a lysate (e.g., a cell lysate) from a host microorganism as a source of the enzymes, or using a plurality of lysates from different host microorganisms as the source of the enzymes.

The reactions of the pathways described herein can be performed in one or more host strains (a) naturally expressing one or more relevant enzymes, (b) genetically engineered to express one or more relevant enzymes, or (c) naturally expressing one or more relevant enzymes and genetically engineered to express one or more relevant enzymes. Alternatively, relevant enzymes can isolated, purified or extracted from of the above types of host cells and used in a purified or semi-purified form. Moreover, such extracts include lysates (e.g. cell lysates) that can be used as sources of relevant enzymes. In the methods provided by the document, all the steps can be performed in host cells, all the steps can be performed using extracted enzymes, or some of the steps can be performed in cells and others can be performed using extracted enzymes.

### Enzymes Generating 6-Hydroxyhexanoate

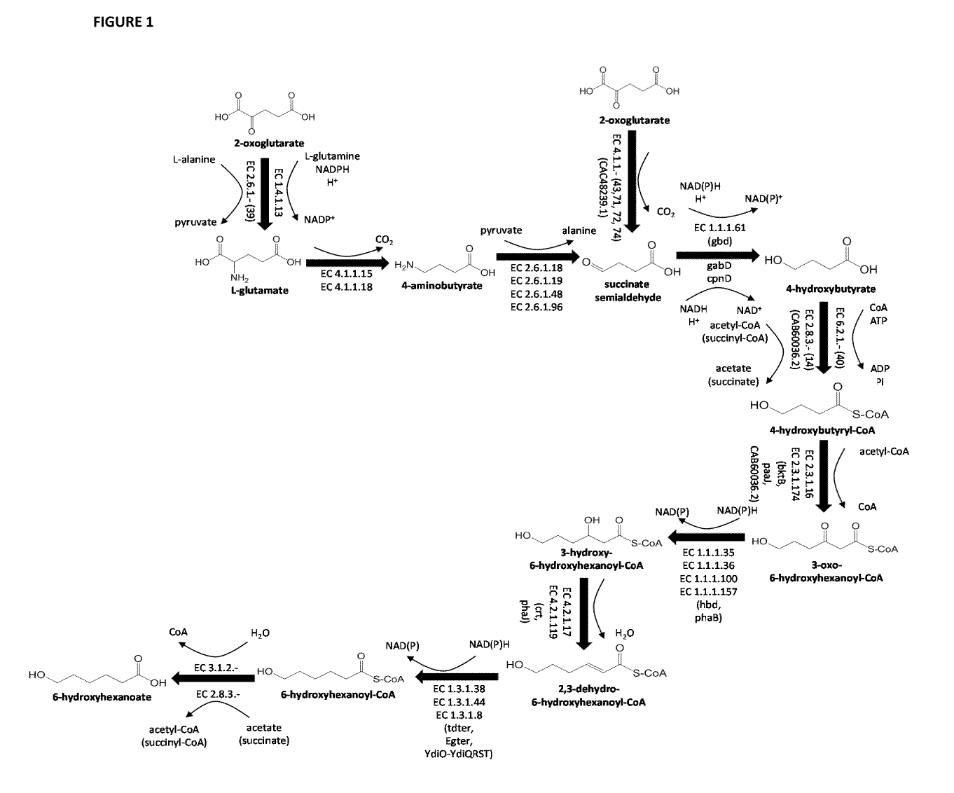

As depicted in FIG. 1, 6-hydroxyhexanaote can be biosynthesized from 2-oxoglutarate through the intermediate 3-oxo-6-hydroxyhexanoyl-CoA, which can be produced from 4-hydroxybutyryl-CoA using a β-ketothiolase. 3-oxo-6-hydroxyhexanoyl-CoA can be converted to 6-hydroxyhexanoate using a 3-hydroxyacyl-CoA dehydrogenase or 3-oxoacyl-CoA dehydrogenase, an enoyl-CoA hydratase, a trans-2-enoyl-CoA reductase, and a thioesterase or a CoA transferase.

In some embodiments, a β-ketothiolase may be classified under EC 2.3.1.16, such as the gene product of bktB or may be classified under EC 2.3.1.174 such as the gene product of paaJ. The β-ketothiolase encoded by bktB from Cupriavidus necator accepts acetyl-CoA and butanoyl-CoA as substrates, forming a CoA-activated C6 aliphatic backbone (see, e.g., Haywood et al., FEMS Microbiology Letters, 1988, 52:91-96; Slater et al., J. Bacteriol., 1998, 180(8):1979-1987). The β-ketothiolase encoded by paaJ from Escherichia coli accepts succinyl-CoA and acetyl-CoA as substrates, forming a CoA-activated backbone (Nogales et al., Microbiology, 2007, 153, 357-365). See, for example, SEQ ID NO:1 and SEQ ID NO:13 in FIG. 6.

In some embodiments, a 3-hydroxyacyl-CoA dehydrogenase or 3-oxoacyl-CoA dehydrogenase can be classified under EC 1.1.1.-. For example, the 3-hydroxyacyl-CoA dehydrogenase can be classified under EC 1.1.1.35, such as the gene product of fadB; classified under EC 1.1.1.157, such as the gene product of hbd (also can be referred to as a 3-hydroxybutyryl-CoA dehydrogenase); or classified under EC 1.1.1.36, such as the acetoacetyl-CoA reductase gene product of phaB (Liu & Chen, Appl. Microbiol. Biotechnol., 2007, 76(5):1153-1159; Shen et al., Appl. Environ. Microbiol., 2011, 77(9):2905-2915; Budde et al., J. Bacteriol., 2010, 192(20):5319-5328).

In some embodiments, a 3-oxoacyl-CoA reductase can be classified under EC 1.1.1.100, such as the gene product of fabG (Budde et al., J. Bacteriol., 2010, 192(20):5319-5328; Nomura et al., Appl. Environ. Microbiol., 2005, 71(8):4297-4306).

In some embodiments, an enoyl-CoA hydratase can be classified under EC 4.2.1.17, such as the gene product of crt, or classified under EC 4.2.1.119, such as the gene product of phaJ (Shen et al., 2011, supra; Fukui et al., J. Bacteriol., 1998, 180(3):667-673).

In some embodiments, a trans-2-enoyl-CoA reductase can be classified under EC 1.3.1.38 or EC 1.3.1.44, such as the gene product of Egter (Nishimaki et al., J. Biochem., 1984, 95:1315-1321; Shen et al., 2011, supra) or tdter (Bond-Watts et al., Biochemistry, 2012, 51:6827-6837) or EC 1.3.1.8 (Inui et al., Eur. J. Biochem., 1984, 142, 121-126).

In some embodiments, the terminal carboxyl group leading to the synthesis of 6-hydroxyhexanoate is enzymatically formed in 6-hydroxyhexanoyl-CoA by a thioesterase classified under EC 3.1.2.-, resulting in the production of 6-hydroxyhexanoate. The thioesterase can be the gene product of YciA or Acot13 (Cantu et al., Protein Science, 2010, 19, 1281-1295; Zhuang et al., Biochemistry, 2008, 47(9):2789-2796; Naggert et al., J. Biol. Chem., 1991, 266(17):11044-11050).

In some embodiments, the terminal carboxyl group leading to the synthesis of 6-hydroxyhexanoate is enzymatically formed in 6-hydroxyhexanoyl-CoA by a CoA-transferase classified under, for example, EC 2.8.3- such as the gene product of cat2 from Clostridium kluyveri, abfT from Clostridium aminobutyricum or the 5-hydroxypentanoate CoA-transferase from Clostridium viride.

### Enzymes Generating the Terminal Carboxyl Groups in the Biosynthesis of Adipic Acid

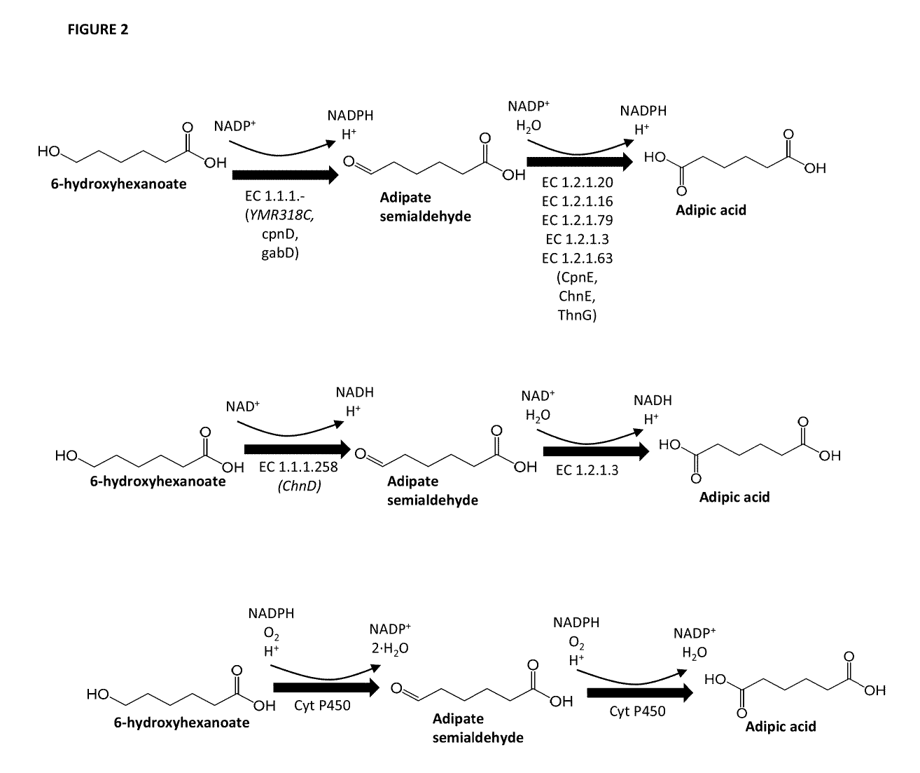

As depicted in FIG. 2, the terminal carboxyl group leading to the production of adipic acid can be enzymatically formed using an aldehyde dehydrogenase, a 5-oxovalerate dehydrogenase, a 6-oxohexanoate dehydrogenase, a 7-oxoheptanoate dehydrogenase, or a monooxygenase.

In some embodiments, the second terminal carboxyl group leading to the synthesis of adipic acid can be enzymatically formed in adipate semialdehyde by an aldehyde dehydrogenase classified under EC 1.2.1.3 (Guerrillot & Vandecasteele, Eur. J. Biochem., 1977, 81, 185-192). See, FIG. 2.

In some embodiments, the second terminal carboxyl group leading to the synthesis of adipic acid is enzymatically formed in adipate semialdehyde by EC 1.2.1.- such as a 5-oxovalerate dehydrogenase classified, for example, under EC 1.2.1.20, such as the gene product of CpnE, a 6-oxohexanoate dehydrogenase classified, for example, EC 1.2.1.63 such as the gene product of ChnE from Acinetobacter sp., or a 7-oxoheptanoate dehydrogenase such as the gene product of ThnG from Sphingomonas macrogolitabida (Iwaki et al., Appl. Environ. Microbiol., 1999, 65(11), 5158-5162; López-Sánchez et al., Appl. Environ. Microbiol., 2010, 76(1), 110-118)). See, FIG. 2.

In some embodiments, the second terminal carboxyl group leading to the synthesis of adipic acid is enzymatically formed in adipate semialdehyde by a monooxygenase in the cytochrome P450 family such as CYP4F3B (see, e.g., Sanders et al., J. Lipid Research, 2005, 46(5):1001-1008; Sanders et al., The FASEB Journal, 2008, 22(6):2064-2071). See, FIG. 2.

### Enzymes Generating the Terminal Amine Groups in the Biosynthesis of Hexamethylenediamine or 6-Aminohexanoate

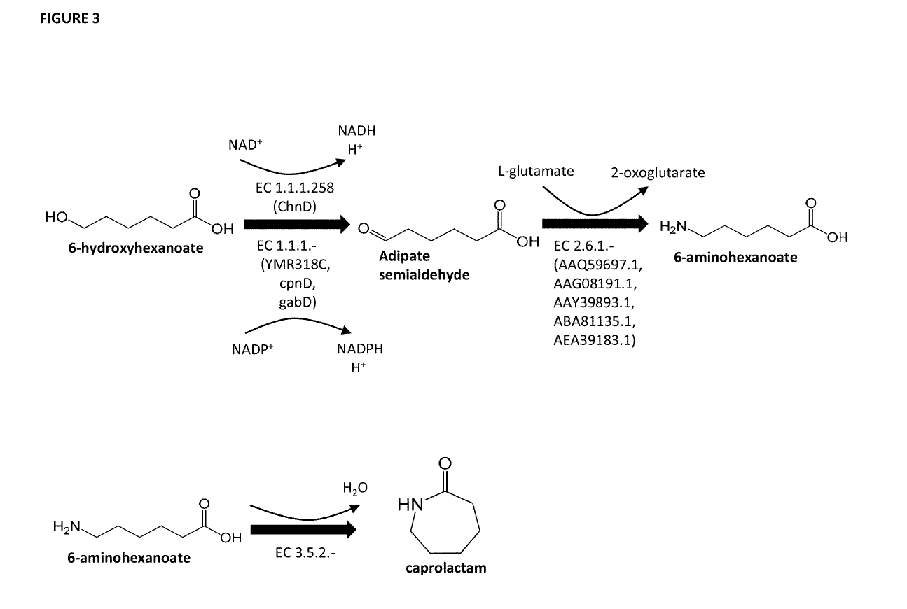

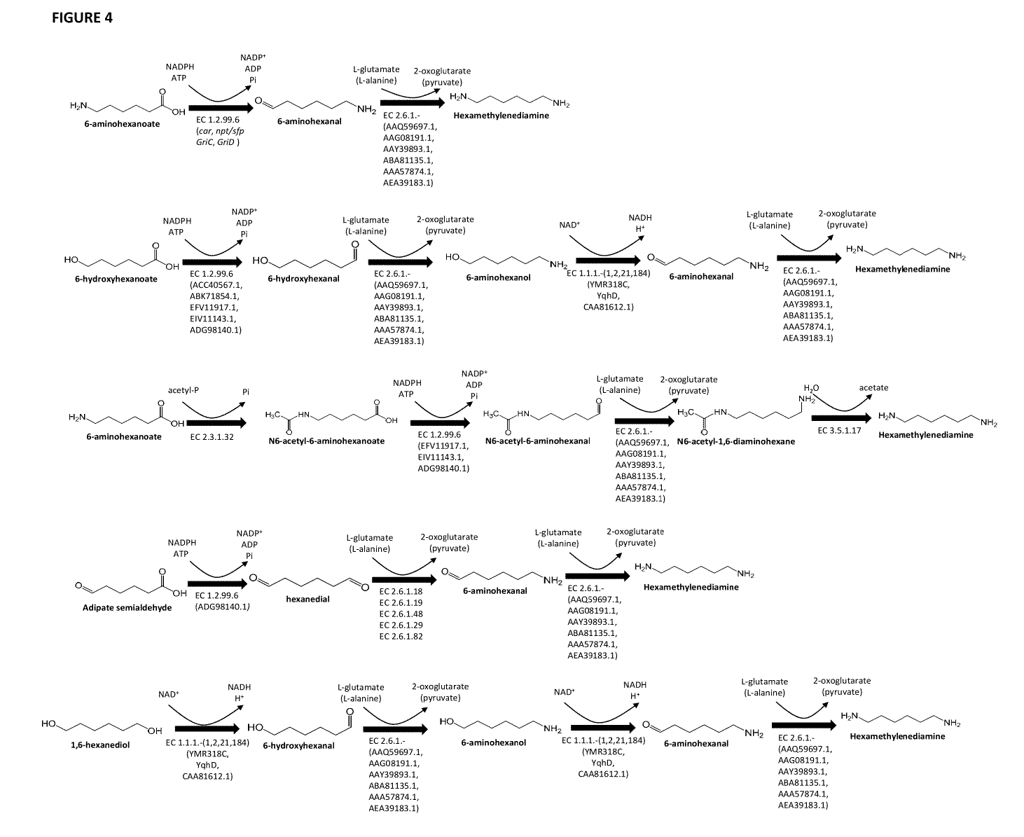

As depicted in FIG. 3 and FIG. 4, terminal amine groups can be enzymatically formed using a ω-transaminase or a deacylase.

In some embodiments, a terminal amine group leading to the synthesis of 6-aminohexanoic acid is enzymatically formed in adipate semialdehyde by a ω-transaminase classified, for example, under EC 2.6.1.-, e.g., EC 2.6.1.18, EC 2.6.1.19, EC 2.6.1.29, EC 2.6.1.48, or EC 2.6.1.82 such as that obtained from Chromobacterium violaceum (Genbank Accession No. AAQ59697.1, SEQ ID NO: 7), Pseudomonas aeruginosa (Genbank Accession No. AAG08191.1, SEQ ID NO: 8), Pseudomonas syringae (Genbank Accession No. AAY39893.1, SEQ ID NO: 9), Rhodobacter sphaeroides (Genbank Accession No. ABA81135.1, SEQ ID NO: 10), Vibrio fluvialis (Genbank Accession No. AEA39183.1, SEQ ID NO: 12), Streptomyces griseus, or Clostridium viride. See, FIG. 3.

An additional ω-transaminase that can be used in the methods and hosts described herein is from Escherichia coli (Genbank Accession No. AAA57874.1, SEQ ID NO: 11). Some of the ω-transaminases classified, for example, under EC 2.6.1.29 or EC 2.6.1.82 are diamine ω-transaminases (e.g., SEQ ID NO: 11).

The reversible ω-transaminase from Chromobacterium violaceum (Genbank Accession No. AAQ59697.1, SEQ ID NO: 7) has demonstrated analogous activity accepting 6-aminohexanoic acid as amino donor, thus forming the first terminal amine group in adipate semialdehyde (Kaulmann et al., Enzyme and Microbial Technology, 2007, 41, 628-637).

The reversible 4-aminobubyrate:2-oxoglutarate transaminase from Streptomyces griseus has demonstrated activity for the conversion of 6-aminohexanoate to adipate semialdehyde (Yonaha et al., Eur. J. Biochem., 1985, 146, 101-106).

The reversible 5-aminovalerate transaminase from Clostridium viride has demonstrated activity for the conversion of 6-aminohexanoate to adipate semialdehyde (Barker et al., J. Biol. Chem., 1987, 262(19), 8994-9003).

In some embodiments, the second terminal amine group leading to the synthesis of hexamethylenediamine is enzymatically formed in 6-aminohexanal by a diamine transaminase classified, for example, under EC 2.6.1.29 or classified, for example, under EC 2.6.1.82, such as the gene product of YgjG from E. coli (Genbank Accession No. AAA57874.1, SEQ ID NO: 12). The transaminases set forth in SEQ ID NOs:7-10 and 11 also can be used to produce hexamethylenediamine. See, FIG. 4.

The gene product of ygjG accepts a broad range of diamine carbon chain length substrates, such as putrescine, cadaverine and spermidine (Samsonova et al., BMC Microbiology, 2003, 3:2).

The diamine transaminase from E. coli strain B has demonstrated activity for 1,7 diaminoheptane (Kim, The Journal of Chemistry, 1964, 239(3), 783-786).

In some embodiments, the second terminal amine group leading to the synthesis of heptamethylenediamine is enzymatically formed in N6-acetyl-1,6-diaminohexane by a deacylase classified, for example, under EC 3.5.1.17 such as an acyl lysine deacylase.

### Enzymes Generating the Terminal Hydroxyl Groups in the Biosynthesis of 1,6 Hexanediol

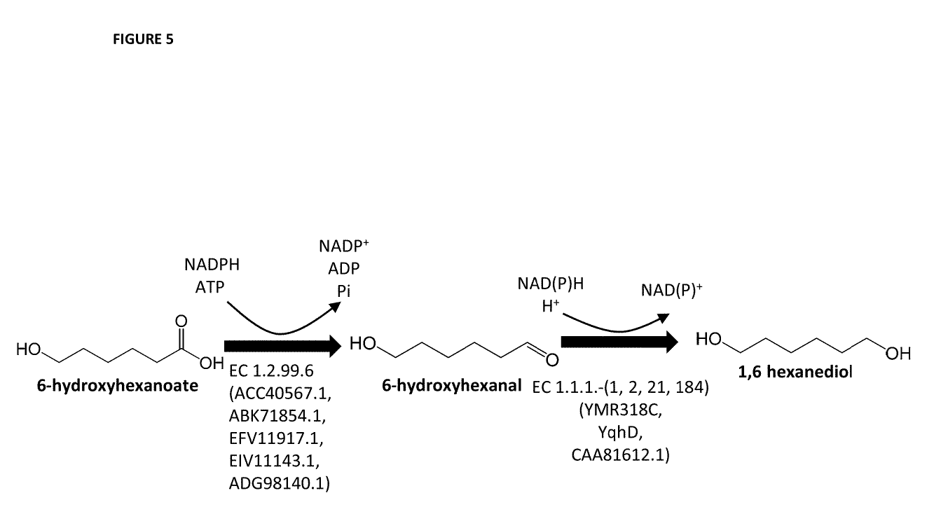

As depicted in FIG. 5, the terminal hydroxyl group can be enzymatically formed using an alcohol dehydrogenase. For example, the second terminal hydroxyl group leading to the synthesis of 1,6 hexanediol can be enzymatically formed in 6-hydroxyhexanal by an alcohol dehydrogenase classified under EC 1.1.1.- (e.g., EC 1.1.1.1, 1.1.1.2, 1.1.1.21, or 1.1.1.184) such as the gene product of YMR318C or YqhD (Liu et al., Microbiology, 2009, 155, 2078-2085; Larroy et al., 2002, Biochem J., 361(Pt 1), 163-172; Jarboe, 2011, Appl. Microbiol. Biotechnol., 89(2), 249-257) or the protein having GenBank Accession No. CAA81612.1.

### Biochemical Pathways

### Pathways to 6-Hydroxyhexanoate

In some embodiments, 6-hydroxyhexanoate is synthesized from the central metabolite, 2-oxoglutarate, by conversion of 2-oxoglutarate to L-glutamate by a glutamate synthase classified, for example, under EC 1.4.1.13 or a α-aminotransferase classified, for example, under EC 2.6.1.- such as EC 2.6.1.39; followed by conversion of L-glutamate to 4-aminobutyrate by a glutamate decarboxylase classified, for example, under EC 4.1.1.15 or EC 4.1.1.18; followed by conversion of 4-aminobutyrate to succinate semialdehyde by a ω-transaminase classified, for example, under EC 2.6.1.18, EC 2.6.1.19, EC 2.6.1.48, or EC 2.6.1.96 such as the gene product of gabT from Escherichia coli (Bartsch et al., J. Bacteriol., 1990, 172(12), 7035).; followed by conversion of succinate semialdehyde to 4-hydroxybutyrate by an alcohol dehydrogenase classified, for example, under EC 1.1.1.61 such as the gene product of gbd (e.g., from Sorangium cellulosum), gabD (Bartsch et al., J. Bacteriol., 1990, 172(12), 7035) or YihU (Saito et al., J. Biol. Chem., 2009, 284(24), 16442-16452), or a 5-hydroxyvalerate dehydrogenase such as the gene product of cpnD (see, for example, Iwaki et al., 2002, Appl. Environ. Microbiol., 68(11):5671-5684); followed by conversion of 4-hydroxybutyrate to 4-hydroxybutyryl-CoA using a CoA-ligase classified under, for example, EC 6.2.1- (e.g., EC 6.2.1.40) or a CoA-transferase classified under, for example, EC 2.8.3.- such as the gene product of cat2 from Clostridium kluyveri, abfT from Clostridium aminobutyricum or the 5-hydroxypentanoate CoA-transferase from Clostridium viride; followed by conversion of 4-hydroxybutyryl-CoA to 3-oxo-6-hydroxyhexanoyl-CoA using a β-ketothiolase classified, for example, under EC 2.3.1.16 or EC 2.3.1.174 such as the gene product of bktB or paaJ (e.g., SEQ ID NO: 1 or 13) or the β-ketothiolase activity encoded by CAB60036.2 (e.g., SEQ ID NO: 14); followed by conversion to 3-hydroxy-6-hydroxyhexanoyl-CoA using a 3-hydroxyacyl-CoA dehydrogenase classified, for example, under EC 1.1.1.- such as EC 1.1.1.35 (e.g., the gene product of fadB), EC 1.1.1.36 (e.g., the gene product of phaB), or EC 1.1.1.157 (e.g., the gene product of hbc1) or a 3-oxoacyl-CoA reductase classified, for example, under EC 1.1.1.100, such as the gene product of fabG; followed by conversion of 3-hydroxy-6-hydroxyhexanoyl-CoA to 2,3-dehydro-6-hydroxyhexanoyl-CoA using an enoyl-CoA hydratase classified, for example, under EC 4.2.1.17 such as the gene product of crt or classified under EC 4.2.1.119 such as the gene product of phaJ; followed by conversion of 2,3-dehydro-6-hydroxyhexanoyl-CoA to 6-hydroxyhexanoyl-CoA by a trans-2-enoyl-CoA reductase classified, for example, under EC 1.3.1.38, EC 1.3.1.44, or EC 1.3.1.8 such as the gene product of Egter or tdter; followed by conversion of 6-hydroxyhexanoyl-CoA to 6-hydroxyhexanoate by a thioesterase classified, for example, under EC 3.1.2.- such as the gene product of YciA or Acot13 or a CoA-transferase classified, for example, under EC 2.8.3.-. See FIG. 1.

In some embodiments, 2-oxoglutarate is converted to succinate semialdehyde using a carboxy-lyase classified, for example, under EC. 4.1.1.- like 2-oxoglutarate decarboxylase classified, for example, under EC 4.1.1.71 or a branch-chain decarboxylase classified, for example, under EC 4.1.1.72 such as the gene product of kdcA or kivD or an indolepyruvate decarboxylase classified, for example, under EC 4.1.1.74 or a phenylpyruvate decarboxylase classified, for example, under EC 4.1.1.43. Succinate semialdehyde produced in this fashion can be converted to 6-hydroxyhexanoate as described above. See, FIG. 1.

### Pathways Using 6-Hydroxyhexanoate as Central Precursor to Adipic Acid

In some embodiments, adipic acid is synthesized from 6-hydroxyhexanoate, by conversion of 6-hydroxyhexanoate to adipate semialdehyde by an alcohol dehydrogenase classified under EC 1.1.1.- such as the gene product of YMR318C (classified, for example, under EC 1.1.1.2, see Genbank Accession No. CAA90836.1) (Larroy et al., 2002, Biochem J., 361(Pt 1), 163-172), cpnD (Iwaki et al., 2002, Appl. Environ. Microbiol., 68(11):5671-5684) or gabD (Lütke-Eversloh & Steinbüchel, 1999, FEMS Microbiology Letters, 181(1):63-71) or a 6-hydroxyhexanoate dehydrogenase classified, for example, under EC 1.1.1.258 such as the gene product of ChnD (Iwaki et al., Appl. Environ. Microbiol., 1999, 65(11):5158-5162); followed by conversion of adipate semialdehyde to adipic acid by a dehydrogenase classified, for example, under EC 1.2.1.- such as a 7-oxoheptanoate dehydrogenase (e.g., the gene product of ThnG), a 6-oxohexanoate dehydrogenase (e.g., the gene product of ChnE), a glutarate semialdehyde dehydrogenase classified, for example, under EC 1.2.1.20, a 5-oxovalerate dehydrogenase such as the gene product of CpnE, or an aldehyde dehydrogenase classified under EC 1.2.1.3. See FIG. 2. The alcohol dehydrogenase encoded by YMR318C has broad substrate specificity, including the oxidation of C6 alcohols.

In some embodiments, adipic acid is synthesized from the central precursor, 6-hydroxyhexanoate, by conversion of 6-hydroxyhexanoate to adipate semialdehyde by a cytochrome P450 (Sanders et al., J. Lipid Research, 2005, 46(5), 1001-1008; Sanders et al., The FASEB Journal, 2008, 22(6), 2064-2071); followed by conversion of adipate semialdehyde to adipic acid by a monooxygenase in the cytochrome P450 family such as CYP4F3B. See FIG. 2.

### Pathway Using 6-Hydroxyhexanoate as Central Precursor to 6-Aminohexanoate and ε-Caprolactam

In some embodiments, 6-aminohexanoate is synthesized from the central precursor, 6-hydroxyhexanoate, by conversion of 6-hydroxyhexanoate to adipate semialdehyde by an alcohol dehydrogenase classified, for example, under EC 1.1.1.2 such as the gene product of YMR318C, a 6-hydroxyhexanoate dehydrogenase classified, for example, under EC 1.1.1.258, a 5-hydroxypentanoate dehydrogenase classified, for example, under EC 1.1.1.- such as the gene product of cpnD, or a 4-hydroxybutyrate dehydrogenase classified, for example, under EC 1.1.1.- such as the gene product of gabD; followed by conversion of adipate semialdehyde to 6-aminohexanoate by a ω-transaminase (EC 2.6.1.18, EC 2.6.1.19, EC 2.6.1.29, EC 2.6.1.48, or EC 2.6.1.82 such as one of SEQ ID NOs:7-10 or 12, see above). See FIG. 3.

In some embodiments, ε-caprolactam is synthesized from the central precursor, 6-hydroxyhexanoate, by conversion of 6-hydroxyhexanoate to adipate semialdehyde by an alcohol dehydrogenase classified, for example, under EC 1.1.1.2 such as the gene product of YMR318C, a 6-hydroxyhexanoate dehydrogenase classified, for example, under EC 1.1.1.258, a 5-hydroxypentanoate dehydrogenase classified, for example, under EC 1.1.1.- such as the gene product of cpnD, or a 4-hydroxybutyrate dehydrogenase classified, for example, under EC 1.1.1.- such as the gene product of gabD; followed by conversion of adipate semialdehyde to 6-aminohexanoate by a ω-transaminase (EC 2.6.1.18, EC 2.6.1.19, EC 2.6.1.29, EC 2.6.1.48, or EC 2.6.1.82); followed by conversion of 6-aminohexanoate to ε-caprolactam by an amidohydrolase (EC 3.5.2.-). See FIG. 3.

In some embodiments, ε-caprolactam is synthesized from the central precursor, 6-aminohexanoate by the last step described above (i.e., by conversion using an amidohydrolase such as one in EC. 3.5.2.-). See FIG. 3.

### Pathway Using 6-Aminohexanoate, 6-Hydroxyhexanoate, Adipate Semialdehyde, or 1,6-Hexanediol as a Central Precursor to Hexamethylenediamine

In some embodiments, hexamethylenediamine is synthesized from the central precursor, 6-aminohexanoate, by conversion of 6-aminohexanoate to 6-aminohexanal by a carboxylate reductase classified, for example, under EC 1.2.99.6 such as the gene product of car in combination with a phosphopantetheine transferase enhancer (e.g., encoded by a sfp gene from Bacillus subtilis or npt gene from Nocardia) or the gene products of GriC and GriD from Streptomyces griseus (Suzuki et al., J. Antibiot., 2007, 60(6), 380-387); followed by conversion of 6-aminohexanal to hexamethylenediamine by a ω-transaminase such as a ω-transaminase in EC 2.6.1.-, (e.g., EC 2.6.1.18, EC 2.6.1.19, EC 2.6.1.48, EC 2.6.1.82 such as SEQ ID NOs:7-12). The carboxylate reductase can be obtained, for example, from Mycobacterium marinum (Genbank Accession No. ACC40567.1, SEQ ID NO: 2), Mycobacterium smegmatis (Genbank Accession No. ABK71854.1, SEQ ID NO: 3), Segniliparus rugosus (Genbank Accession No. EFV11917.1, SEQ ID NO: 4), Mycobacterium massiliense (Genbank Accession No. EIV11143.1, SEQ ID NO: 5), or Segniliparus rotundus (Genbank Accession No. ADG98140.1, SEQ ID NO: 6). See FIG. 4.

The carboxylate reductase encoded by the gene product of car and enhancer npt or sfp has broad substrate specificity, including terminal difunctional C4 and C5 carboxylic acids (Venkitasubramanian et al., Enzyme and Microbial Technology, 2008, 42, 130-137).

In some embodiments, hexamethylenediamine is synthesized from the central precursor, 6-hydroxyhexanoate (which can be produced as described in FIG. 1), by conversion of 6-hydroxyhexanoate to 6-hydroxyhexanal by a carboxylate reductase classified, for example, under EC 1.2.99.6 such as the gene product of car (see above) in combination with a phosphopantetheine transferase enhancer (e.g., encoded by a sfp gene from Bacillus subtilis or npt gene from Nocardia) or the gene product of GriC & GriD (Suzuki et al., 2007, supra); followed by conversion of 6-aminohexanal to 6-aminohexanol by a ω-transaminase classified, for example, under EC 2.6.1.18, EC 2.6.1.19, EC 2.6.1.29, EC 2.6.1.48, or EC 2.6.1.82 such as SEQ ID NOs:7-12, see above; followed by conversion to 7-aminoheptanal by an alcohol dehydrogenase classified, for example, under EC 1.1.1.- (e.g., EC 1.1.1.1, EC 1.1.1.2, EC 1.1.1.21, or EC 1.1.1.184) such as the gene product of YMR318C or YqhD (Liu et al., Microbiology, 2009, 155, 2078-2085; Larroy et al., 2002, Biochem J., 361(Pt 1), 163-172; Jarboe, 2011, Appl. Microbiol. Biotechnol., 89(2), 249-257) or the protein having GenBank Accession No. CAA81612.1; followed by conversion to heptamethylenediamine by a ω-transaminase classified, for example, under EC 2.6.1.18, EC 2.6.1.19, EC 2.6.1.29, EC 2.6.1.48, or EC 2.6.1.82 such as SEQ ID NOs:7-12, see above. See FIG. 4.

In some embodiments, hexamethylenediamine is synthesized from the central precursor, 6-aminohexanoate, by conversion of 6-aminohexanoate to N6-acetyl-6-aminohexanoate by an N-acetyltransferase such as a lysine N-acetyltransferase classified, for example, under EC 2.3.1.32; followed by conversion to N6-acetyl-6-aminohexanal by a carboxylate reductase classified, for example, under EC 1.2.99.6 such as the gene product of car (see above, e.g., SEQ ID NO: 4, 5, or 6) in combination with a phosphopantetheine transferase enhancer (e.g., encoded by a sfp gene from Bacillus subtilis or npt gene from Nocardia) or the gene product of GriC & GriD; followed by conversion to N6-acetyl-1,6-diaminohexane by a ω-transaminase classified, for example, under EC 2.6.1.18, EC 2.6.1.19, EC 2.6.1.29, EC 2.6.1.48, or EC 2.6.1.82 such as SEQ ID NOs:7-12, see above; followed by conversion to heptamethylenediamine by an acyl lysine deacylase classified, for example, under EC 3.5.1.17. See, FIG. 4.

In some embodiments, hexamethylenediamine is synthesized from the central precursor, adipate semialdehyde, by conversion of adipate semialdehyde to hexanedial by a carboxylate reductase classified, for example, under EC 1.2.99.6 such as the gene product of car (see above, e.g., SEQ ID NO:6) in combination with a phosphopantetheine transferase enhancer (e.g., encoded by a sfp gene from Bacillus subtilis or npt gene from Nocardia) or the gene product of GriC & GriD; followed by conversion to 6-aminohexanal by a ω-transaminase classified, for example, under EC 2.6.1.18, EC 2.6.1.19, EC 2.6.1.29, EC 2.6.1.48, or EC 2.6.1.82; followed by conversion to hexamethylenediamine by a ω-transaminase classified, for example, under EC 2.6.1.18, EC 2.6.1.19, EC 2.6.1.29, EC 2.6.1.48, or EC 2.6.1.82 such as SEQ ID NOs:7-12. See FIG. 4.

In some embodiments, hexamethylenediamine is synthesized from 1,6-hexanediol by conversion of 1,6-hexanedion to 6-hydroxyhexanal using an alcohol dehydrogenase classified, for example, under EC 1.1.1.- (e.g., EC 1.1.1.1, EC 1.1.1.2, EC 1.1.1.21, or EC 1.1.1.184) such as the gene product of YMR318C or YqhD or the protein having GenBank Accession No. CAA81612.1; followed by conversion to 6-aminohexanol by a ω-transaminase classified, for example, under EC 2.6.1.18, EC 2.6.1.19, EC 2.6.1.29, EC 2.6.1.48, or EC 2.6.1.82 such as SEQ ID NOs:7-12, followed by conversion to 6-aminohexanal by an alcohol dehydrogenase classified, for example, under EC 1.1.1.- (e.g., EC 1.1.1.1, EC 1.1.1.2, EC 1.1.1.21, or EC 1.1.1.184) such as the gene product of YMR318C or YqhD or the protein having GenBank Accession No. CAA81612.1, followed by conversion to hexamethylenediamine by a ω-transaminase classified, for example, under EC 2.6.1.18, EC 2.6.1.19, EC 2.6.1.29, EC 2.6.1.48, or EC 2.6.1.82 such as SEQ ID NOs:7-12. See FIG. 4.

### Pathways Using 6-Hydroxyhexanoate as Central Precursor to 1,6-Hexanediol

In some embodiments, 1,6 hexanediol is synthesized from the central precursor, 6-hydroxyhexanoate, by conversion of 6-hydroxyhexanoate to 6-hydroxyhexanal by a carboxylate reductase classified, for example, under EC 1.2.99.6 such as the gene product of car (see above, e.g., SEQ ID NO: 2, 3, 4, 5, or 6) in combination with a phosphopantetheine transferase enhancer (e.g., encoded by a sfp gene from Bacillus subtilis or npt gene from Nocardia) or the gene products of GriC and GriD from Streptomyces griseus (Suzuki et al., J. Antibiot., 2007, 60(6), 380-387); followed by conversion of 6-hydroxyhexanal to 1,6 hexanediol by an alcohol dehydrogenase (classified, for example, under EC 1.1.1.- such as EC 1.1.1.1, EC 1.1.1.2, EC 1.1.1.21, or EC 1.1.1.184) such as the gene product of YMR318C or YqhD (from E. coli, GenBank Accession No. AAA69178.1) (see, e.g., Liu et al., Microbiology, 2009, 155, 2078-2085; Larroy et al., 2002, Biochem J., 361(Pt 1), 163-172; or Jarboe, 2011, Appl. Microbiol. Biotechnol., 89(2), 249-257) or the protein having GenBank Accession No. CAA81612.1 (from Geobacillus stearothermophilus). See, FIG. 5.

### Cultivation Strategy

In some embodiments, one or more C6 building blocks are biosynthesized in a recombinant host using anaerobic, aerobic or micro-aerobic cultivation conditions. In some embodiments, the cultivation strategy entails nutrient limitation such as nitrogen, phosphate or oxygen limitation.

In some embodiments, a cell retention strategy using, for example, ceramic membranes can be employed to achieve and maintain a high cell density during either fed-batch or continuous fermentation.

In some embodiments, the principal carbon source fed to the fermentation in the synthesis of one or more C6 building blocks can derive from biological or non-biological feedstocks.

In some embodiments, the biological feedstock can be or can derive from, monosaccharides, disaccharides, lignocellulose, hemicellulose, cellulose, lignin, levulinic acid and formic acid, triglycerides, glycerol, fatty acids, agricultural waste, condensed distillers' solubles, or municipal waste.

The efficient catabolism of crude glycerol stemming from the production of biodiesel has been demonstrated in several microorganisms such as Escherichia coli, Cupriavidus necator, Pseudomonas oleavorans, Pseudomonas putida and Yarrowia lipolytica (Lee et al., Appl. Biochem. Biotechnol., 2012, 166:1801-1813; Yang et al., Biotechnology for Biofuels, 2012, 5:13; Meijnen et al., Appl. Microbiol. Biotechnol., 2011, 90:885-893).

The efficient catabolism of lignocellulosic-derived levulinic acid has been demonstrated in several organisms such as Cupriavidus necator and Pseudomonas putida in the synthesis of 3-hydroxyvalerate via the precursor propanoyl-CoA (Jaremko and Yu, 2011, supra; Martin and Prather, J. Biotechnol., 2009, 139:61-67).

The efficient catabolism of lignin-derived aromatic compounds such as benzoate analogues has been demonstrated in several microorganisms such as Pseudomonas putida, Cupriavidus necator (Bugg et al., Current Opinion in Biotechnology, 2011, 22, 394-400; Perez-Pantoja et al., FEMS Microbiol. Rev., 2008, 32, 736-794).

The efficient utilization of agricultural waste, such as olive mill waste water has been demonstrated in several microorganisms, including Yarrowia lipolytica (Papanikolaou et al., Bioresour. Technol., 2008, 99(7):2419-2428).

The efficient utilization of fermentable sugars such as monosaccharides and disaccharides derived from cellulosic, hemicellulosic, cane and beet molasses, cassava, corn and other agricultural sources has been demonstrated for several microorganism such as Escherichia coli, Corynebacterium glutamicum and Lactobacillus delbrueckii and Lactococcus lactis (see, e.g., Hermann et al, J. Biotechnol., 2003, 104:155-172; Wee et al., Food Technol. Biotechnol., 2006, 44(2):163-172; Ohashi et al., J. Bioscience and Bioengineering, 1999, 87(5):647-654).

The efficient utilization of furfural, derived from a variety of agricultural lignocellulosic sources, has been demonstrated for Cupriavidus necator (Li et al., Biodegradation, 2011, 22:1215-1225).

In some embodiments, the non-biological feedstock can be or can derive from natural gas, syngas, CO₂/H₂, methanol, ethanol, benzoate, non-volatile residue (NVR) or a caustic wash waste stream from cyclohexane oxidation processes, or terephthalic acid/isophthalic acid mixture waste streams.

The efficient catabolism of methanol has been demonstrated for the methylotrophic yeast Pichia pastoris.

The efficient catabolism of ethanol has been demonstrated for Clostridium kluyveri (Seedorf et al., Proc. Natl. Acad. Sci. USA, 2008, 105(6) 2128-2133).

The efficient catabolism of CO₂ and H₂, which may be derived from natural gas and other chemical and petrochemical sources, has been demonstrated for Cupriavidus necator (Prybylski et al., Energy, Sustainability and Society, 2012, 2:11).

The efficient catabolism of syngas has been demonstrated for numerous microorganisms, such as Clostridium ljungdahlii and Clostridium autoethanogenum (Kopke et al., Applied and Environmental Microbiology, 2011, 77(15):5467-5475).

The efficient catabolism of the non-volatile residue waste stream from cyclohexane processes has been demonstrated for numerous microorganisms, such as Delftia acidovorans and Cupriavidus necator (Ramsay et al., Applied and Environmental Microbiology, 1986, 52(1):152-156).

In some embodiments, the host microorganism is a prokaryote. For example, the prokaryote can be a bacterium from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia such as Delftia acidovorans; from the genus Bacillus such as Bacillus subtillis; from the genus Lactobacillus such as Lactobacillus delbrueckii; or from the genus Lactococcus such as Lactococcus lactis. Such prokaryotes also can be a source of genes to construct recombinant host cells described herein that are capable of producing one or more C6 building blocks.

In some embodiments, the host microorganism is a eukaryote. For example, the eukaryote can be a filamentous fungus, e.g., one from the genus Aspergillus such as Aspergillus niger. Alternatively, the eukaryote can be a yeast, e.g., one from the genus Saccharomyces such as Saccharomyces cerevisiae; from the genus Pichia such as Pichia pastoris; or from the genus Yarrowia such as Yarrowia lipolytica; from the genus Issatchenkia such as Issathenkia orientalis; from the genus Debaryomyces such as Debaryomyces hansenii; from the genus Arxula such as Arxula adenoinivorans; or from the genus Kluyveromyces such as Kluyveromyces lactis. Such eukaryotes also can be a source of genes to construct recombinant host cells described herein that are capable of producing one or more C6 building blocks.

### Metabolic Engineering

The present document provides methods involving less than all the steps described for all the above pathways. Such methods can involve, for example, one, two, three, four, five, six, seven, eight, nine, ten, eleven, twelve or more of such steps. Where less than all the steps are included in such a method, the first, and in some embodiments the only, step can be any one of the steps listed.

Furthermore, recombinant hosts described herein can include any combination of the above enzymes such that one or more of the steps, e.g., one, two, three, four, five, six, seven, eight, nine, ten, or more of such steps, can be performed within a recombinant host. This document provides host cells of any of the genera and species listed and genetically engineered to express one or more (e.g., two, three, four, five, six, seven, eight, nine, 10, 11, 12 or more) recombinant forms of any of the enzymes recited in the document. Thus, for example, the host cells can contain exogenous nucleic acids encoding enzymes catalyzing one or more of the steps of any of the pathways described herein.

In addition, this document recognizes that where enzymes have been described as accepting CoA-activated substrates, analogous enzyme activities associated with [acp]-bound substrates exist that are not necessarily in the same enzyme class.

Also, this document recognizes that where enzymes have been described accepting (R)-enantiomers of substrate, analogous enzyme activities associated with (S)-enantiomer substrates exist that are not necessarily in the same enzyme class.

This document also recognizes that where an enzyme is shown to accept a particular co-factor, such as NADPH, or co-substrate, such as acetyl-CoA, many enzymes are promiscuous in terms of accepting a number of different co-factors or co-substrates in catalyzing a particular enzyme activity. Also, this document recognizes that where enzymes have high specificity for e.g., a particular co-factor such as NADH, an enzyme with similar or identical activity that has high specificity for the co-factor NADPH may be in a different enzyme class.

In some embodiments, the enzymes in the pathways outlined herein are the result of enzyme engineering via non-direct or rational enzyme design approaches with aims of improving activity, improving specificity, reducing feedback inhibition, reducing repression, improving enzyme solubility, changing stereo-specificity, or changing co-factor specificity.

In some embodiments, the enzymes in the pathways outlined here can be gene dosed, i.e., overexpressed, into the resulting genetically modified organism via episomal or chromosomal integration approaches.

In some embodiments, genome-scale system biology techniques such as Flux Balance Analysis can be utilized to devise genome scale attenuation or knockout strategies for directing carbon flux to a C6 building block.

Attenuation strategies include, but are not limited to; the use of transposons, homologous recombination (double cross-over approach), mutagenesis, enzyme inhibitors and RNAi interference.

In some embodiments, fluxomic, metabolomic and transcriptomal data can be utilized to inform or support genome-scale system biology techniques, thereby devising genome scale attenuation or knockout strategies in directing carbon flux to a C6 building block.

In some embodiments, the host microorganism's tolerance to high concentrations of a C6 building block can be improved through continuous cultivation in a selective environment.

In some embodiments, the host microorganism's endogenous biochemical network can be attenuated or augmented to (1) ensure the intracellular availability of acetyl-CoA and 4-hydroxybutyryl-CoA, (2) create an NADH or NADPH imbalance that may only be balanced via the formation of one or more C6 building blocks, (3) prevent degradation of central metabolites, central precursors leading to and including one or more C6 building blocks and/or (4) ensure efficient efflux from the cell.

In some embodiments requiring intracellular availability of acetyl-CoA for C6 building block synthesis, endogenous enzymes catalyzing the hydrolysis of acetyl-CoA such as short-chain length thioesterases can be attenuated in the host organism.

In some embodiments requiring the intracellular availability of acetyl-CoA for C6 building block synthesis, an endogenous phosphotransacetylase generating acetate such as pta can be attenuated (Shen et al., Appl. Environ. Microbiol., 2011, 77(9):2905-2915).

In some embodiments requiring the intracellular availability of acetyl-CoA for C6 building block synthesis, an endogenous gene in an acetate synthesis pathway encoding an acetate kinase, such as ack, can be attenuated.

In some embodiments requiring the intracellular availability of acetyl-CoA and NADH for C6 building block synthesis, an endogenous gene encoding an enzyme that catalyzes the degradation of pyruvate to lactate such as lactate dehydrogenase encoded by ldhA can be attenuated (Shen et al., 2011, supra).

In some embodiments, enzymes that catalyze anapleurotic reactions such as PEP carboxylase and/or pyruvate carboxylase can be overexpressed in the host organism.

In some embodiments requiring the intracellular availability of acetyl-CoA and NADH for C6 building block synthesis, endogenous genes encoding enzymes, such as menaquinol-fumarate oxidoreductase, that catalyze the degradation of phophoenolpyruvate to succinate such as frdBC can be attenuated (see, e.g., Shen et al., 2011, supra).

In some embodiments requiring the intracellular availability of acetyl-CoA and NADH for C6 building block synthesis, an endogenous gene encoding an enzyme that catalyzes the degradation of acetyl-CoA to ethanol such as the alcohol dehydrogenase encoded by adhE can be attenuated (Shen et al., 2011, supra).

In some embodiments, where pathways require excess NADH co-factor for C6 building block synthesis, a recombinant formate dehydrogenase gene can be overexpressed in the host organism (Shen et al., 2011, supra).

In some embodiments, where pathways require excess NADH co-factor for C6 building block synthesis, a recombinant NADH-consuming transhydrogenase can be attenuated.

In some embodiments, an endogenous gene encoding an enzyme that catalyzes the degradation of pyruvate to ethanol such as pyruvate decarboxylase can be attenuated.

In some embodiments requiring the intracellular availability of acetyl-CoA for C6 building block synthesis, a recombinant acetyl-CoA synthetase such as the gene product of acs can be overexpressed in the microorganism (Satoh et al., J. Bioscience and Bioengineering, 2003, 95(4):335-341).

In some embodiments, carbon flux can be directed into the pentose phosphate cycle to increase the supply of NADPH by attenuating an endogenous glucose-6-phosphate isomerase (EC 5.3.1.9).

In some embodiments, carbon flux can be redirected into the pentose phosphate cycle to increase the supply of NADPH by overexpression a 6-phosphogluconate dehydrogenase and/or a transketolase (Lee et al., 2003, Biotechnology Progress, 19(5), 1444-1449).

In some embodiments, where pathways require excess NADPH co-factor in the synthesis of a C6 building block, a gene such as UdhA encoding a puridine nucleotide transhydrogenase can be overexpressed in the host organisms (Brigham et al., Advanced Biofuels and Bioproducts, 2012, Chapter 39, 1065-1090).

In some embodiments, where pathways require excess NADPH co-factor in the synthesis of a C6 Building Block, a recombinant glyceraldehyde-3-phosphate-dehydrogenase gene such as GapN can be overexpressed in the host organisms (Brigham et al., 2012, supra).

In some embodiments, where pathways require excess NADPH co-factor in the synthesis of a C6 building block, a recombinant malic enzyme gene such as maeA or maeB can be overexpressed in the host organisms (Brigham et al., 2012, supra).

In some embodiments, where pathways require excess NADPH co-factor in the synthesis of a C6 building block, a recombinant glucose-6-phosphate dehydrogenase gene such as zwf can be overexpressed in the host organisms (Lim et al., J. Bioscience and Bioengineering, 2002, 93(6), 543-549).

In some embodiments, where pathways require excess NADPH co-factor in the synthesis of a C6 building block, a recombinant fructose 1,6 diphosphatase gene such as fbp can be overexpressed in the host organisms (Becker et al., J. Biotechnol., 2007, 132:99-109).

In some embodiments, where pathways require excess NADPH co-factor in the synthesis of a C6 building block, endogenous triose phosphate isomerase (EC 5.3.1.1) can be attenuated.

In some embodiments, where pathways require excess NADPH co-factor in the synthesis of a C6 building block, a recombinant glucose dehydrogenase such as the gene product of gdh can be overexpressed in the host organism (Satoh et al., J. Bioscience and Bioengineering, 2003, 95(4):335-341).

In some embodiments, endogenous enzymes facilitating the conversion of NADPH to NADH can be attenuated, such as the NADH generation cycle that may be generated via inter-conversion of glutamate dehydrogenases classified under EC 1.4.1.2 (NADH-specific) and EC 1.4.1.4 (NADPH-specific).

In some embodiments, an endogenous glutamate dehydrogenase (EC 1.4.1.3) that utilizes both NADH and NADPH as co-factors can be attenuated.

In some embodiments, a membrane-bound cytochrome P450 such as CYP4F3B can be solubilized by only expressing the cytosolic domain and not the N-terminal region that anchors the P450 to the endoplasmic reticulum (Scheller et al., J. Biol. Chem., 1994, 269(17):12779-12783).

In some embodiments, an enoyl-CoA reductase can be solubilized via expression as a fusion protein with a small soluble protein, for example, the maltose binding protein (Gloerich et al., FEBS Letters, 2006, 580, 2092-2096).

In some embodiments using hosts that naturally accumulate polyhydroxyalkanoates, the endogenous polymer synthase enzymes can be attenuated in the host strain.

In some embodiments, a L-alanine dehydrogenase can be overexpressed in the host to regenerate L-alanine from pyruvate as an amino donor for ω-transaminase reactions.

In some embodiments, a L-glutamate dehydrogenase, a L-glutamine synthetase, or a glutamate synthase can be overexpressed in the host to regenerate L-glutamate from 2-oxoglutarate as an amino donor for ω-transaminase reactions.

In some embodiments, enzymes such as a pimeloyl-CoA dehydrogenase classified under, EC 1.3.1.62; an acyl-CoA dehydrogenase classified, for example, under EC 1.3.8.7, EC 1.3.8.1, or EC 1.3.99.-; and/or a butyryl-CoA dehydrogenase classified, for example, under EC 1.3.8.6 that degrade central metabolites and central precursors leading to and including C6 building blocks can be attenuated.

In some embodiments, endogenous enzymes activating C6 building blocks via Coenzyme A esterification such as CoA-ligases (e.g., an adipyl-CoA synthetase) classified under, for example, EC 6.2.1.- can be attenuated.

In some embodiments, the efflux of a C6 building block across the cell membrane to the extracellular media can be enhanced or amplified by genetically engineering structural modifications to the cell membrane or increasing any associated transporter activity for a C6 building block.

The efflux of hexamethylenediamine can be enhanced or amplified by overexpressing broad substrate range multidrug transporters such as Blt from Bacillus subtilis (Woolridge et al., 1997, J. Biol. Chem., 272(14):8864-8866); AcrB and AcrD from Escherichia coli (Elkins & Nikaido, 2002, J. Bacteriol., 184(23), 6490-6499), NorA from Staphylococcus aereus (Ng et al., 1994, Antimicrob Agents Chemother, 38(6), 1345-1355), or Bmr from Bacillus subtilis (Neyfakh, 1992, Antimicrob Agents Chemother, 36(2), 484-485).

The efflux of 6-aminohexanoate and heptamethylenediamine can be enhanced or amplified by overexpressing the solute transporters such as the lysE transporter from Corynebacterium glutamicum (Bellmann et al., 2001, Microbiology, 147, 1765-1774).

The efflux of adipic acid can be enhanced or amplified by overexpressing a dicarboxylate transporter such as the SucE transporter from Corynebacterium glutamicum (Huhn et al., Appl. Microbiol. & Biotech., 89(2), 327-335).

### Producing C6 Building Blocks Using a Recombinant Host

Typically, one or more C6 building blocks can be produced by providing a host microorganism and culturing the provided microorganism with a culture medium containing a suitable carbon source as described above. In general, the culture media and/or culture conditions can be such that the microorganisms grow to an adequate density and produce a C6 building block efficiently. For large-scale production processes, any method can be used such as those described elsewhere (Manual of Industrial Microbiology and Biotechnology, 2^{nd} Edition, Editors: A. L. Demain and J. E. Davies, ASM Press; and Principles of Fermentation Technology, P. F. Stanbury and A. Whitaker, Pergamon). Briefly, a large tank (e.g., a 100 gallon, 200 gallon, 500 gallon, or more tank) containing an appropriate culture medium is inoculated with a particular microorganism. After inoculation, the microorganism is incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank.

Once transferred, the microorganisms can be incubated to allow for the production of a C6 building block. Once produced, any method can be used to isolate C6 building blocks. For example, C6 building blocks can be recovered selectively from the fermentation broth via adsorption processes. In the case of adipic acid and 6-aminoheptanoic acid, the resulting eluate can be further concentrated via evaporation, crystallized via evaporative and/or cooling crystallization, and the crystals recovered via centrifugation. In the case of hexamethylenediamine and 1,6-hexanediol, distillation may be employed to achieve the desired product purity.

The invention will be further described in the following examples, which do not limit the scope of the invention described in the claims.

### EXAMPLES

### Example 1

### Enzyme Activity of ω-Transaminase Using Adipate Semialdehyde as Substrate and Forming 6-Aminohexanoate

A nucleotide sequence encoding a His-tag was added to the nucleic acid sequences from Chromobacterium violaceum, Pseudomonas aeruginosa, Pseudomonas syringae, Rhodobacter sphaeroides, and Vibrio fluvialis encoding the ω-transaminases of SEQ ID NOs: 7, 8, 9, 10 and 12, respectively (see FIG. 6) such that N-terminal HIS tagged ω-transaminases could be produced. Each of the resulting modified genes was cloned into a pET21a expression vector under control of the T7 promoter and each expression vector was transformed into a BL21[DE3] E. coli host. The resulting recombinant E. coli strains were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., 6-aminohexanoate to adipate semialdehyde) were performed in a buffer composed of a final concentration of 50 mM HEPES buffer (pH=7.5), 10 mM 6-aminohexanoate, 10 mM pyruvate and 100 μM pyridoxyl 5′ phosphate. Each enzyme activity assay reaction was initiated by adding cell free extract of the ω-transaminase gene product or the empty vector control to the assay buffer containing the 6-aminohexanoate and incubated at 25° C. for 24 h, with shaking at 250 rpm. The formation of L-alanine from pyruvate was quantified via RP-HPLC.

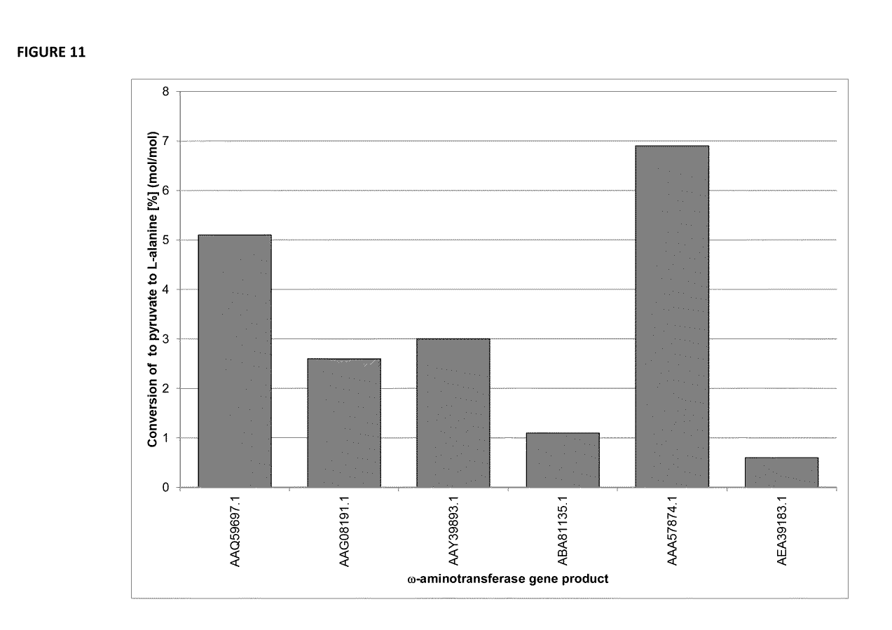

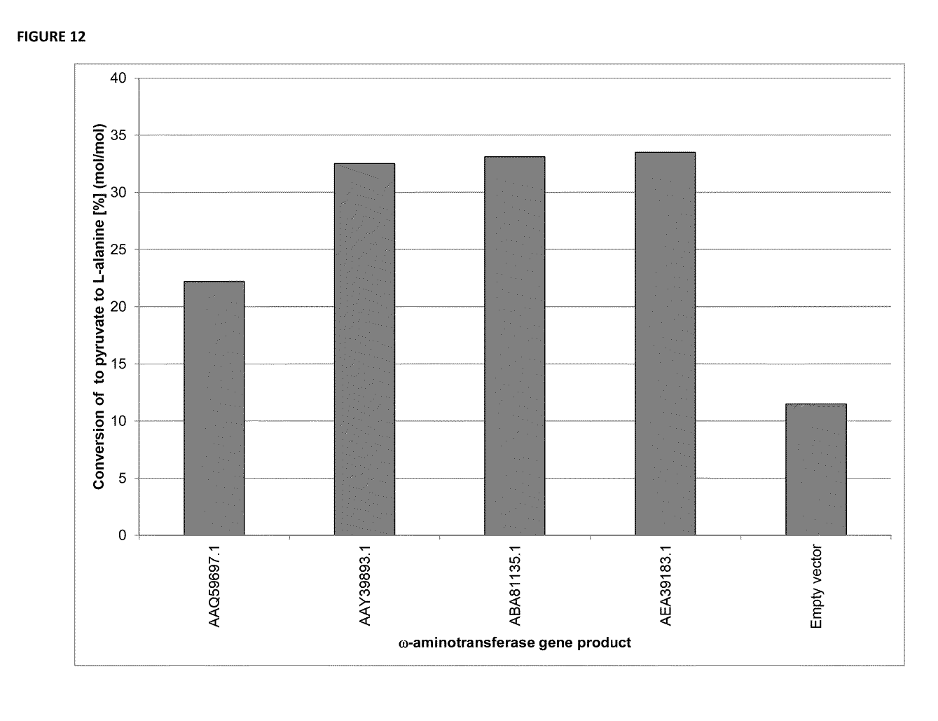

Each enzyme only control without 6-aminoheptanoate demonstrated low base line conversion of pyruvate to L-alanine See FIG. 11. The gene product of SEQ ID NO 7, SEQ ID NO 9, SEQ ID NO 10 and SEQ ID NO 12 accepted 6-aminohexanote as substrate as confirmed against the empty vector control. See FIG. 12.

Enzyme activity in the forward direction (i.e., adipate semialdehyde to 6-aminohexanoate) was confirmed for the transaminases of SEQ ID NO 7, SEQ ID NO 8, SEQ ID NO 9, SEQ ID NO 10 and SEQ ID NO 12. Enzyme activity assays were performed in a buffer composed of a final concentration of 50 mM HEPES buffer (pH=7.5), 10 mM adipate semialdehyde, 10 mM L-alanine and 100 μM pyridoxyl 5′ phosphate. Each enzyme activity assay reaction was initiated by adding a cell free extract of the ω-transaminase gene product or the empty vector control to the assay buffer containing the adipate semialdehyde and incubated at 25° C. for 4 h, with shaking at 250 rpm. The formation of pyruvate was quantified via RP-HPLC.

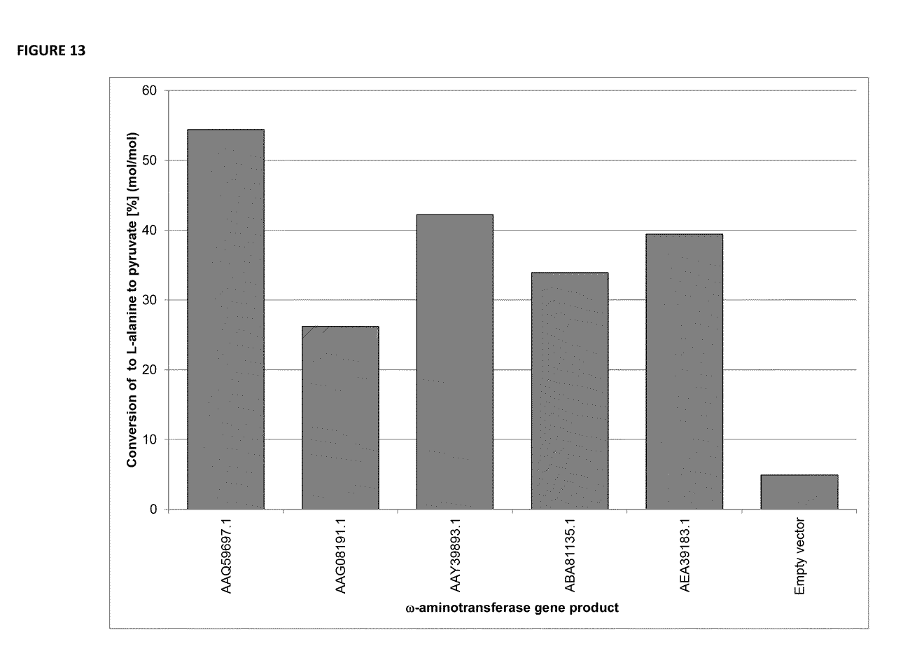

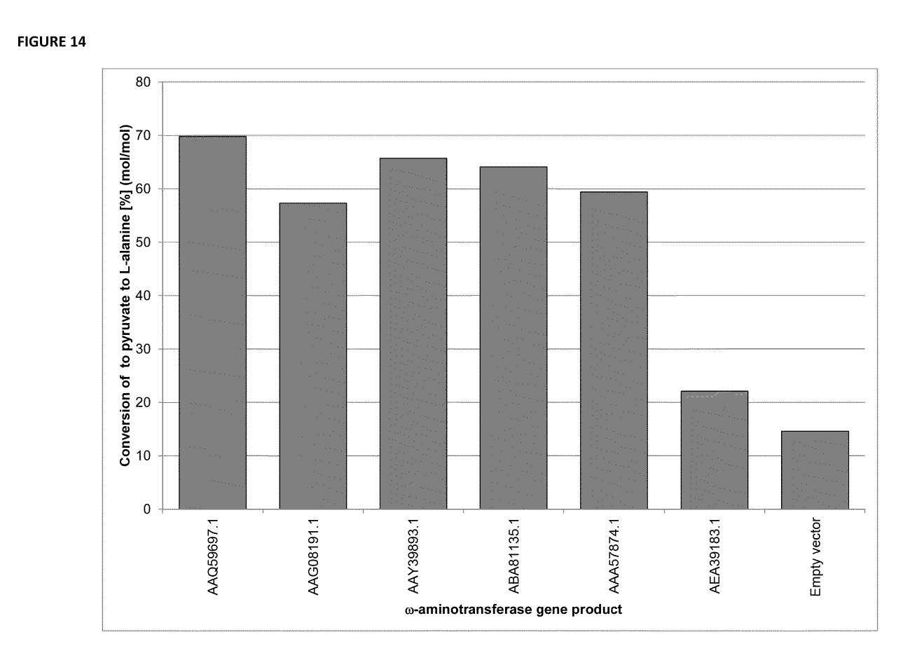

The gene product of SEQ ID NO 7, SEQ ID NO 8, SEQ ID NO 9, SEQ ID NO 10 and SEQ ID NO 12 accepted adipate semialdehyde as substrate as confirmed against the empty vector control. See FIG. 13. The reversibility of the ω-transaminase activity was confirmed, demonstrating that the ω-transaminases of SEQ ID NO 7, SEQ ID NO 8, SEQ ID NO 9, SEQ ID NO 10 and SEQ ID NO 12 accepted adipate semialdehyde as substrate and synthesized 6-aminohexanoate as a reaction product.

### Example 2

### Enzyme Activity of Carboxylate Reductase Using 6-Hydroxyhexanoate as Substrate and Forming 6-Hydroxyhexanal

A nucleotide sequence encoding a His-tag was added to the nucleic acid sequences from Mycobacterium marinum, Mycobacterium smegmatis, Mycobacterium smegmatis, Segniliparus rugosus, Mycobacterium massiliense, and Segniliparus rotundus that encode the carboxylate reductases of SEQ ID NOs: 2-6, respectively (see FIG. 6) such that N-terminal HIS tagged carboxylate reductases could be produced. Each of the modified genes was cloned into a pET Duet expression vector alongside a sfp gene encoding a His-tagged phosphopantetheine transferase from Bacillus subtilis, both under control of the T7 promoter. Each expression vector was transformed into a BL21[DE3] E. coli host along with the expression vectors from Example 3. Each resulting recombinant E. coli strain was cultivated at 37° C. in a 250 mL shake flask culture containing 50 mL LB media and antibiotic selection pressure, with shaking at 230 rpm. Each culture was induced overnight at 37° C. using an auto-induction media.

The pellet from each induced shake flask culture was harvested via centrifugation. Each pellet was resuspended and lysed via sonication. The cell debris was separated from the supernatant via centrifugation. The carboxylate reductases and phosphopantetheine transferase were purified from the supernatant using Ni-affinity chromatography, diluted 10-fold into 50 mM HEPES buffer (pH=7.5) and concentrated via ultrafiltration.

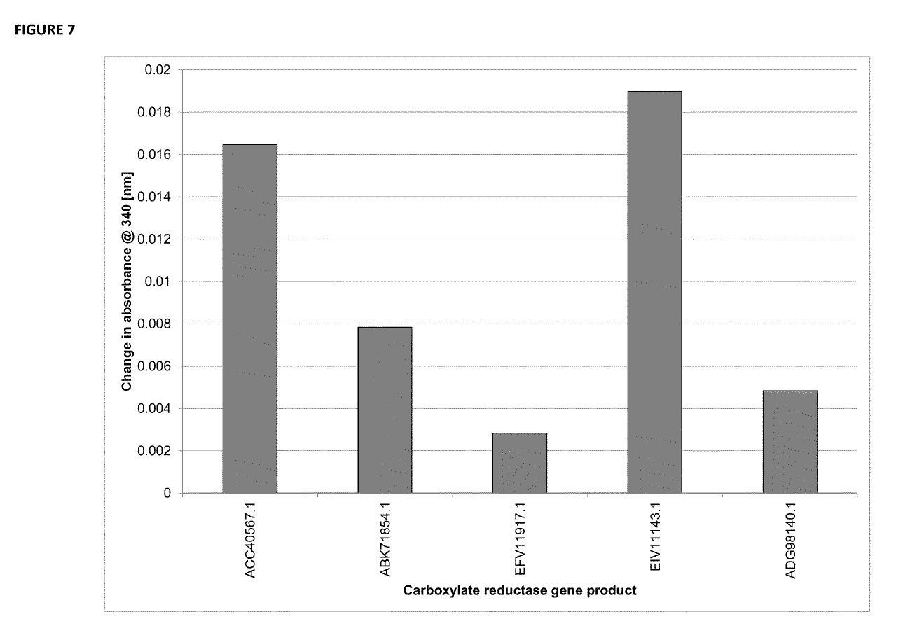

Enzyme activity (i.e., 6-hydroxyhexanoate to 6-hydroxyhexanal) assays were performed in triplicate in a buffer composed of a final concentration of 50 mM HEPES buffer (pH=7.5), 2 mM 6-hydroxyhexanal, 10 mM MgCl₂, 1 mM ATP, and 1 mM NADPH. Each enzyme activity assay reaction was initiated by adding purified carboxylate reductase and phosphopantetheine transferase or the empty vector control to the assay buffer containing the 6-hydroxyhexanoate and then incubated at room temperature for 20 min. The consumption of NADPH was monitored by absorbance at 340 nm. Each enzyme only control without 6-hydroxyhexanoate demonstrated low base line consumption of NADPH. See FIG. 7.

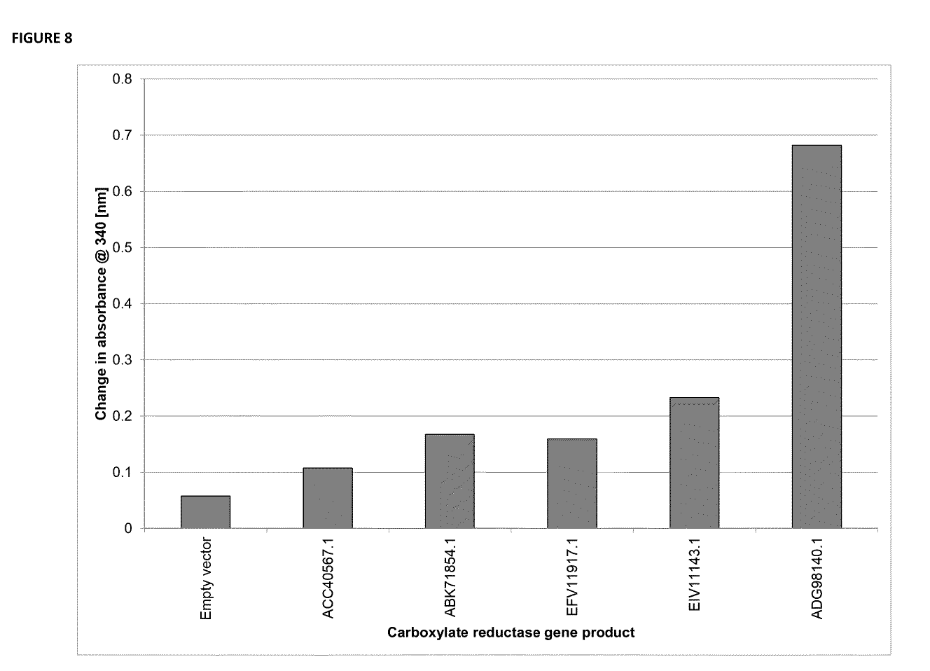

The gene products of SEQ ID NO 2-6, enhanced by the gene product of sfp, accepted 6-hydroxyhexanoate as substrate as confirmed against the empty vector control (see FIG. 8), and synthesized 6-hydroxyhexanal.

### Example 3

### Enzyme Activity of ω-Transaminase for 6-Aminohexanol, Forming 6-Oxohexanol

A nucleotide sequence encoding an N-terminal His-tag was added to the Chromobacterium violaceum, Pseudomonas aeruginosa, Pseudomonas syringae, Rhodobacter sphaeroides, Escherichia coli, and Vibrio fluvialis nucleic acid sequences encoding the ω-transaminases of SEQ ID NOs: 7-12, respectively (see FIG. 6) such that N-terminal HIS tagged ω-transaminases could be produced. The modified genes were cloned into a pET21a expression vector under the T7 promoter. Each expression vector was transformed into a BL21[DE3] E. coli host. Each resulting recombinant E. coli strain were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., 6-aminohexanol to 6-oxohexanol) were performed in a buffer composed of a final concentration of 50 mM HEPES buffer (pH=7.5), 10 mM 6-aminohexanol, 10 mM pyruvate, and 100 μM pyridoxyl 5′ phosphate. Each enzyme activity assay reaction was initiated by adding cell free extract of the ω-transaminase gene product or the empty vector control to the assay buffer containing the 6-aminohexanol and then incubated at 25° C. for 4 h, with shaking at 250 rpm. The formation of L-alanine was quantified via RP-HPLC.

Each enzyme only control without 6-aminohexanol had low base line conversion of pyruvate to L-alanine See FIG. 11.

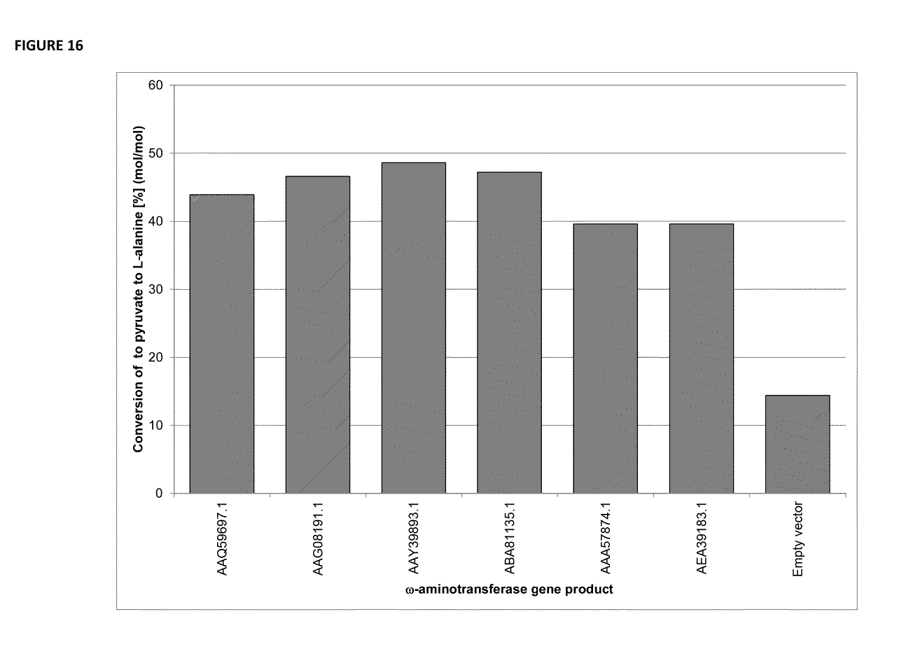

The gene products of SEQ ID NOs: 7-12 accepted 6-aminohexanol as substrate as confirmed against the empty vector control (see FIG. 16) and synthesized 6-oxohexanol as reaction product. Given the reversibility of the ω-transaminase activity (see Example 1), it can be concluded that the gene products of SEQ ID NOs: 7-12 accept 6-aminohexanol as substrate and form 6-oxohexanol.

### Example 4

### Enzyme Activity of ω-Transaminase Using Hexamethylenediamine as Substrate and Forming 6-Aminohexanal

A nucleotide sequence encoding an N-terminal His-tag was added to the Chromobacterium violaceum, Pseudomonas aeruginosa, Pseudomonas syringae, Rhodobacter sphaeroides, Escherichia coli, and Vibrio fluvialis nucleic acid sequences encoding the ω-transaminases of SEQ ID NOs: 7-12, respectively (see FIG. 6) such that N-terminal HIS tagged ω-transaminases could be produced. The modified genes were cloned into a pET21a expression vector under the T7 promoter. Each expression vector was transformed into a BL21[DE3] E. coli host. Each resulting recombinant E. coli strain were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., hexamethylenediamine to 6-aminohexanal) were performed in a buffer composed of a final concentration of 50 mM HEPES buffer (pH=7.5), 10 mM hexamethylenediamine, 10 mM pyruvate, and 100 μM pyridoxyl 5′ phosphate. Each enzyme activity assay reaction was initiated by adding cell free extract of the ω-transaminase gene product or the empty vector control to the assay buffer containing the hexamethylenediamine and then incubated at 25° C. for 4 h, with shaking at 250 rpm. The formation of L-alanine was quantified via RP-HPLC.

Each enzyme only control without hexamethylenediamine had low base line conversion of pyruvate to L-alanine See FIG. 11.

The gene products of SEQ ID NO 7-12 accepted hexamethylenediamine as substrate as confirmed against the empty vector control and synthesized 6-aminohexanal as reaction product. Given the reversibility of the ω-transaminase activity (see Example 1), it can be concluded that the gene products of SEQ ID NOs: 7-12 accept 6-aminohexanal as substrate and form hexamethylenediamine.

### Example 5

### Enzyme Activity of Carboxylate Reductase for N6-Acetyl-6-Aminohexanoate, Forming N6-Acetyl-6-Aminohexanal

The activity of each of the N-terminal His-tagged carboxylate reductases of SEQ ID NOs: 4-6 (see Example 2, and FIG. 6) for converting N6-acetyl-6-aminohexanoate to N6-acetyl-6-aminohexanal was assayed in triplicate in a buffer composed of a final concentration of 50 mM HEPES buffer (pH=7.5), 2 mM N6-acetyl-6-aminohexanoate, 10 mM MgCl₂, 1 mM ATP, and 1 mM NADPH. The assays were initiated by adding purified carboxylate reductase and phosphopantetheine transferase or the empty vector control to the assay buffer containing the N6-acetyl-6-aminohexanoate then incubated at room temperature for 20 min. The consumption of NADPH was monitored by absorbance at 340 nm. Each enzyme only control without N6-acetyl-6-aminohexanoate demonstrated low base line consumption of NADPH. See FIG. 7.

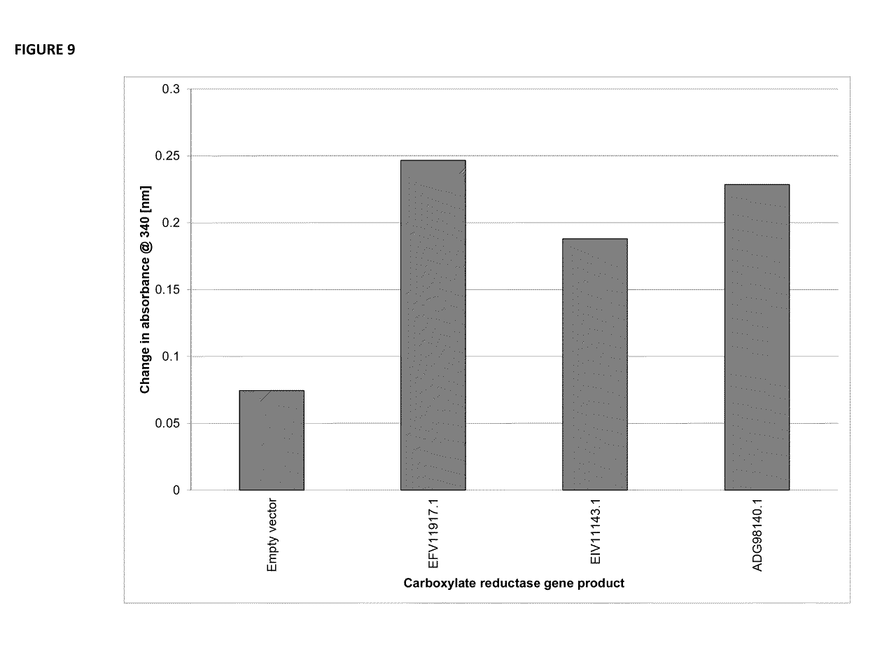

The gene products of SEQ ID NO 4-6, enhanced by the gene product of sfp, accepted N6-acetyl-6-aminohexanoate as substrate as confirmed against the empty vector control (see FIG. 9), and synthesized N6-acetyl-6-aminohexanal.

### Example 6

### Enzyme Activity of ω-Transaminase Using N6-Acetyl-1,6-Diaminohexane, and Forming N6-Acetyl-6-Aminohexanal

The activity of the N-terminal His-tagged ω-transaminases of SEQ ID NOs: 7-12 (see Example 4, and FIG. 6) for converting N6-acetyl-1,6-diaminohexane to N6-acetyl-6-aminohexanal was assayed using a buffer composed of a final concentration of 50 mM HEPES buffer (pH=7.5), 10 mM N6-acetyl-1,6-diaminohexane, 10 mM pyruvate and 100 μM pyridoxyl 5′ phosphate. Each enzyme activity assay reaction was initiated by adding a cell free extract of the ω-transaminase or the empty vector control to the assay buffer containing the N6-acetyl-1,6-diaminohexane then incubated at 25° C. for 4 h, with shaking at 250 rpm. The formation of L-alanine was quantified via RP-HPLC.

Each enzyme only control without N6-acetyl-1,6-diaminohexane demonstrated low base line conversion of pyruvate to L-alanine See FIG. 11.

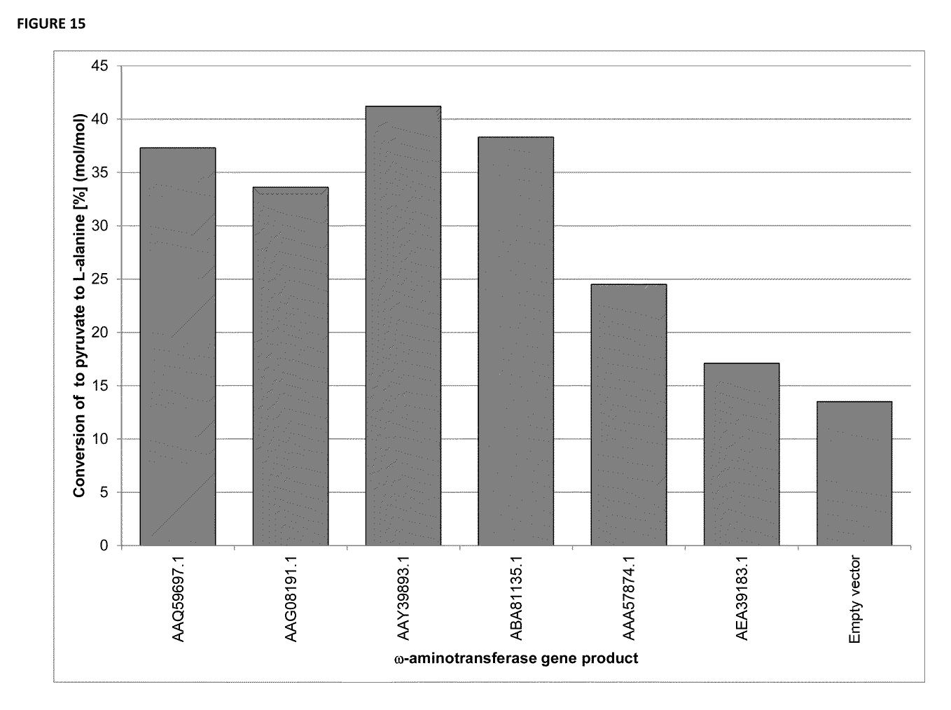

The gene product of SEQ ID NO 7-12 accepted N6-acetyl-1,6-diaminohexane as substrate as confirmed against the empty vector control (see FIG. 15) and synthesized N6-acetyl-6-aminohexanal as reaction product.

Given the reversibility of the ω-transaminase activity (see example 1), the gene products of SEQ ID NOs: 7-12 accept N6-acetyl-6-aminohexanal as substrate forming N6-acetyl-1,6-diaminohexane.

### Example 7

### Enzyme Activity of Carboxylate Reductase Using Adipate Semialdehyde as Substrate and Forming Hexanedial

The N-terminal His-tagged carboxylate reductase of SEQ ID NO 6 (see Example 2 and FIG. 6) was assayed using adipate semialdehyde as substrate. The enzyme activity assay was performed in triplicate in a buffer composed of a final concentration of 50 mM HEPES buffer (pH=7.5), 2 mM adipate semialdehyde, 10 mM MgCl₂, 1 mM ATP and 1 mM NADPH. The enzyme activity assay reaction was initiated by adding purified carboxylate reductase and phosphopantetheine transferase or the empty vector control to the assay buffer containing the adipate semialdehyde and then incubated at room temperature for 20 min. The consumption of NADPH was monitored by absorbance at 340 nm. The enzyme only control without adipate semialdehyde demonstrated low base line consumption of NADPH. See FIG. 7.

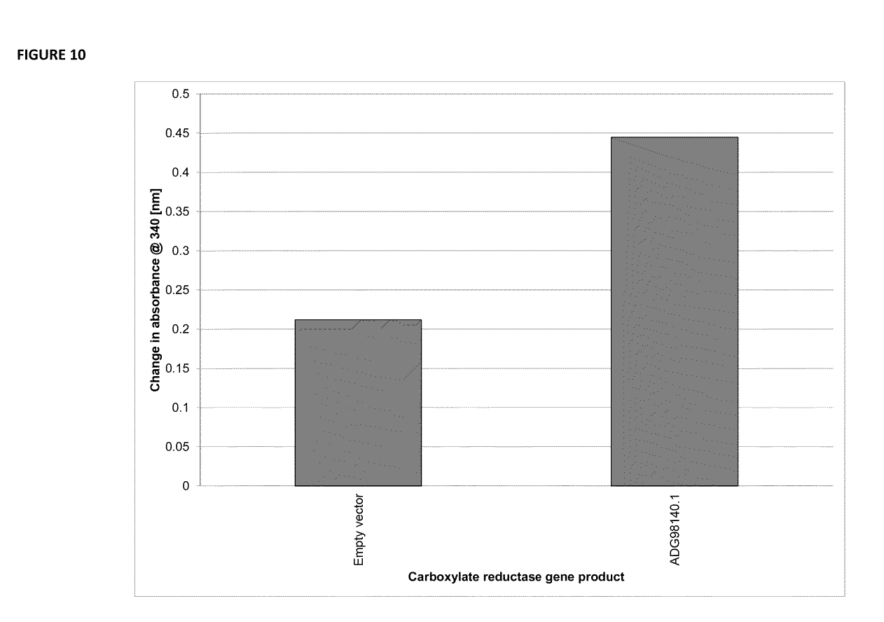

The gene product of SEQ ID NO: 6, enhanced by the gene product of sfp, accepted adipate semialdehyde as substrate as confirmed against the empty vector control (see FIG. 10) and synthesized hexanedial.

### Example 8

### β-Ketothiolase Activity Using 4-Hydroxybutyryl-CoA and Acetyl-CoA as Substrates and Forming 3-Oxo-6-Hydroxyhexanoyl-CoA

A nucleotide sequence encoding a N-terminal His-tag was added to the gene from Clostridium aminobutyricum encoding the β-ketothiolase activity of SEQ ID NO: 14 (see FIG. 6) such that a N-terminal HIS tagged enzyme could be produced. The resulting modified gene was cloned into a pET15b expression vector under control of the T7 promoter and the expression vector was transformed into a BL21[DE3] E. coli host. The resulting recombinant E. coli strain was cultivated at 37° C. in a 1 L shake flask culture containing 350 mL LB media and ampicillin antibiotic selection pressure, with shaking at 230 rpm. Each culture was induced overnight at 25° C. using 1 mM IPTG.

The pellet from each induced shake flask culture was harvested via centrifugation. Each pellet was resuspended and lysed via sonication. The cell debris was separated from the supernatant via centrifugation. The enzyme was purified from the supernatant using Ni-affinity chromatography, buffer exchanged and concentrated into 50 mM potassium phosphate buffer (pH=6.8) via ultrafiltration.

Enzyme activity assays converting 4-hydroxybutyryl-CoA and acetyl-CoA to 3-oxo-6-hydroxyhexanoyl-CoA were performed in triplicate in a buffer composed of a final concentration of 50 mM potassium phosphate buffer (pH=6.8), 75 μM ZnCl₂, 10 mM γ-butyrolactone and 5 mM acetyl-CoA. The enzyme activity assay reaction was initiated by adding SEQ ID NO: 14 and lactonase encoded by ChnC from Acinetobacter sp. to final concentrations of 5 [μM] respectively to the assay buffer containing the 10 mM γ-butyrolactone and 5 mM acetyl-CoA and incubated at 30° C. for 3 hour, with shaking at 180 rpm. The formation of 3-oxo-6-hydroxyhexanoyl-CoA was determined via LC-MS.

Negative controls omitting one substrate or one enzyme demonstrated no conversion to 3-oxo-6-hydroxyhexanoyl-CoA. SEQ ID NO: 14 accepted 4-hydroxybutyryl-CoA and acetyl-CoA as substrates and synthesized 3-oxo-6-hydroxyhexanoyl-CoA as reaction product as confirmed via LC-MS.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing 3-oxo-6-hydroxyhexanoyl-CoA or a salt thereof, said method comprising enzymatically converting 4-hydroxybutyryl-CoA to 3-oxo-6-hydroxyhexanoyl-CoA using a polypeptide having β-ketothiolase activity classified under EC. 2.3.1.-.

2. The method of claim 1, wherein said polypeptide having β-ketothiolase activity has at least 70% sequence identity to the amino acid sequence set forth in SEQ ID NOs:1, 13 or 14 or is any other polypeptide having β-ketothiolase activity classified under EC 2.3.1.16 or EC 2.3.1.174.

3.-4. (canceled)

5. The method of claim 1, further comprising enzymatically converting 3-oxo-6-hydroxyhexanoyl-CoA to 6-hydroxyhexanoate using a 3-hydroxyacyl-CoA dehydrogenase or a 3-oxoacyl-CoA reductase, an enoyl-CoA hydratase, a trans-2-enoyl-CoA reductase, and a thioesterase or a CoA transferase.

6. The method of claim 5, wherein:
(a) said 3-hydroxyacyl-CoA dehydrogenase or said 3-oxoacyl-CoA reductase is classified under EC 1.1.1.35, EC 1.1.1.36, EC 1.1.1.100, or EC 1.1.1.157;
(b) said enol-CoA hydratase is classified under EC 4.2.1.17 or EC 4.2.1.119; and/or
(c) said trans-2-enol-CoA reductase is classified under EC 1.3.1.38, EC 1.3.1.44, or EC 1.3.1.8.

7.-8. (canceled)

9. A method for biosynthesizing 6-hydroxyhexanoate, said method comprising enzymatically synthesizing 3-oxo-6-hydroxyhexanoyl-CoA from 4-hydroxybutyryl-CoA using a polypeptide having β-ketothiolase activity classified under EC. 2.3.1.- and enzymatically converting 3-oxo-6-hydroxyhexanoyl-CoA to 6-hydroxyhexanoate.

10. The method of claim 9, wherein 3-oxo-6-hydroxyhexanoyl-CoA is converted to 3-hydroxy-6-hydroxyhexanoyl-CoA using a 3-hydroxyacyl-CoA dehydrogenase or a 3-oxoacyl-CoA reductase, 3-hydroxy-6-hydroxyhexanoyl-CoA is converted to 2,3-dehydro-6-hydroxyhexanoyl-CoA using an enoyl-CoA hydratase, 2,3-dehydro-6-hydroxyhexanoyl-CoA is converted to 6-hydroxyhexanoyl-CoA using a trans-2-enoyl-CoA reductase, and 6-hydroxyhexanoyl-CoA is converted to 6-hydroxyhexanoate using a thioesterase or a CoA transferase.

11. The method of claim 5 or claim 9, said method further comprising enzymatically converting 6-hydroxyhexanoate to adipic acid, 6-aminohexanoate, caprolactam, hexamethylenediamine, or 1,6-hexanediol in one or more steps.

12. The method of claim 11, wherein:
(a) 6-hydroxyhexanoate is converted to adipic acid using one or more of a monooxygenase, an alcohol dehydrogenase, a 4-hydroxybutanoate dehydrogenase, a 5-hydroxyvalerate dehydrogenase, a 6-hydroxyhexanoate dehydrogenase, a 7-oxoheptanoate dehydrogenase, a 6-oxohexanoate dehydrogenase, a 5-oxovalerate dehydrogenase, or an aldehyde dehydrogenase;
(b) 6-hydroxyhexanoate is converted to 6-aminohexanoate using one or more of an alcohol dehydrogenase, a 6-hydroxyhexanoate dehydrogenase, a 5-hydroxypentanoate dehydrogenase, a 4-hydroxybutyrate dehydrogenase and a ω-transaminase;
(c) 6-hydroxyhexanoate is converted to caprolactam using one or more of an alcohol dehydrogenase, a 6-hydroxyhexanoate dehydrogenase, a 5-hydroxypentanoate dehydrogenase, a 4-hydroxybutyrate dehydrogenase; a ω-transaminase and an amidohydrolase;
(d) 6-hydroxyhexanoate is converted to hexamethylenediamine using one or more of a carboxylate reductase, a ω-transaminase, an alcohol dehydrogenase, an N-acetyltransferase, and an acetylputrescine deacylase; and/or
(e) 6-hydroxyhexanoate is converted to 1,6-hexanediol using a carboxylate reductase and an alcohol dehydrogenase.

13.-15. (canceled)

16. The method of claim 12, wherein:
(a) said ω-transaminase has at least 70% sequence identity to any one of the amino acid sequences set forth in SEQ ID NOs. 7-12; and/or
(b) said carboxylate reductase has at least 70% sequence identity to any one of the amino acid sequences set forth in SEQ ID NOs. 2-6.

17.-18. (canceled)

19. The method of claim 1 or claim 9, wherein said 4-hydroxybutyryl-CoA is enzymatically produced from 2-oxoglutarate.

20. The method of claim 19, wherein 4-hydroxybutyryl-CoA is enzymatically produced from 2-oxoglutarate using one or more of a glutamate synthase; a 2-oxoglutarate decarboxylase; a branch chain decarboxylase; a glutamate decarboxylase; a ω-transaminase; a CoA transferase, a CoA ligase, and an alcohol dehydrogenase.

21. The method of claim 1 or claim 9, wherein said method is performed in a recombinant host.

22. The method of claim 21, wherein:
(a) said host is subjected to a cultivation strategy under aerobic, anaerobic or, micro-aerobic cultivation conditions;
(b) said host is cultured under conditions of nutrient limitation;
(c) said host is retained using a ceramic membrane to maintain a high cell density during fermentation;
(d) the principal carbon source fed to the fermentation derives from a biological feedstock; and/or
(e) the principal carbon source fed to the fermentation derives from a non-biological feedstock.

23.-25. (canceled)

26. The method of claim 22, wherein the biological feedstock is, or derives from, monosaccharides, disaccharides, lignocellulose, hemicellulose, cellulose, lignin, levulinic acid, formic acid, triglycerides, glycerol, fatty acids, agricultural waste, condensed distillers' solubles, or municipal waste.

27. (canceled)

28. The method of claim 22, wherein the non-biological feedstock is, or derives from, natural gas, syngas, CO₂/H₂, methanol, ethanol, benzoate, non-volatile residue (NVR) caustic wash waste stream from cyclohexane oxidation processes, or terephthalic acid/isophthalic acid mixture waste streams.

29. The method of claim 21, wherein the host is a prokaryote or a eukaryote.

30. The method of claim 29, wherein said prokaryote is from a genus selected from the group consisting of Escherichia, Clostridia, Corynebacteria, Cupriavidus, Pseudomonas, Delftia, Bacilluss, Lactobacillus, Lactococcus, and Rhodococcus.

31. The method of claim 30, wherein said prokaryote is selected from the group consisting of Escherichia coli, Clostridium ljungdahlii, Clostridium autoethanogenum, Clostridium kluyveri, Corynebacterium glutamicum, Cupriavidus necator, Cupriavidus metallidurans, Pseudomonas fluorescens, Pseudomonas putida, Pseudomonas oleavorans, Delftia acidovorans, Bacillus subtillis, Lactobacillus delbrueckii, Lactococcus lactis, and Rhodococcus equi.

32. (canceled)

33. The method of claim 29, wherein said eukaryote is from a genus selected from the group consisting of Aspergillus, Saccharomyces, Pichia, Yarrowia, Issatchenkia, Debaryomyces, Arxula, and Kluyveromyces.

34. The method of claim 33, wherein said eukaryote is selected from the group consisting of Aspergillus niger, Saccharomyces cerevisiae, Pichia pastoris, Yarrowia lipolytica, Issathenkia orientalis, Debaryomyces hansenii, Arxula adenoinivorans, and Kluyveromyces lactis.

35. The method of claim 21, wherein:
(a) the host's tolerance to high concentrations of a C6 building block is improved through continuous cultivation in a selective environment;
(b) said host comprises an attenuation to one or more of the following enzymes: a polyhydroxyalkanoate synthase, an acetyl-CoA thioesterase, a phosphotransacetylase forming acetate, an acetate kinase, a lactate dehydrogenase, a menaquinol-fumarate oxidoreductase, an alcohol dehydrogenase forming ethanol, a triose phosphate isomerase, a pyruvate decarboxylase, a glucose-6-phosphate isomerase, an NADH-consuming transhydrogenase, an NADH-specific qlutamate dehydrogenase, an NADH/NADPH-utilizing glutamate dehydrogenase, a pimeloyl-CoA dehydrogenase, an acyl-CoA dehydrogenase accepting C6 building blocks and central precursors as substrates, a butaryl-CoA dehydrogenase, or an adipyl-CoA synthetase; and/or
(c) said host overexpresses one or more genes encoding: an acetyl-CoA-synthetase, a 6-phosphogluconate dehydrogenase, a transketolase, a puridine nucleotide transhydrogenase, a gylceraldehyde-3P-dehydrogenase, a malic enzyme, a glucose-6-phosphate dehydrogenase, a glucose dehydrogenase, a fructose 1,6 diphosphatase, a L-alanine dehydrogenase, a L-glutamate dehydrogenase, a formate dehydrogenase, a L-glutamine synthetase, a diamine transporter a dicarboxylate transporter, and/or a multidrug transporter.

36.-37. (canceled)

38. A recombinant host comprising at least one exogenous nucleic acid encoding (i) a β-ketothiolase, (ii) a thioesterase or a CoA transferase, and one or more of (iii) a 3-hydroxyacyl-CoA dehydrogenase or a 3-oxoacyl-CoA reductase, (iv) an enoyl-CoA hydratase, and (v) a trans-2-enoyl-CoA reductase, said host producing 6-hydroxyhexanoate.

39. The recombinant host of claim 38, wherein:
(a) said host further comprising one or more of the following exogenous enzymes: a monooxygenase, an alcohol dehydrogenase, a 4-hydroxybutanoate dehydrogenase, a 5-hydroxyvalerate dehydrogenase, a 6-hydroxyhexanoate dehydrogenase, a 7-oxoheptanoate dehydrogenase, a 6-oxohexanoate dehydrogenase, a 5-oxovalerate dehydrogenase, or an aldehyde dehydrogenase, said host further producing adipic acid;
(b) said host further comprising one or more of the following exogenous enzymes: a monooxygenase, a transaminase, a 6-hydroxyhexanoate dehydrogenase, a 5-hydroxypentanoate dehydrogenase, a 4-hydroxybutyrate dehydrogenase, and an alcohol dehydrogenase said host further producing 8-aminohexanoate;
(c) said host further comprising one or more of the following exogenous enzymes: a monooxygenase, a transaminase, a 6-hydroxyhexanoate dehydrogenase, a 5-hydroxypentanoate dehydrogenase, a 4-hydroxybutyrate dehydrogenase, an alcohol dehydrogenase, and an amidohydrolase, said host further producing caprolactam;
(d) said host further comprising one or more of the following exogenous enzymes: a carboxylate reductase, a ω-transaminase, a deacylase, an N-acetyl transferase, or an alcohol dehydrogenase, said host further producing hexamethylenediamine; and/or
(e) said host further comprising an exogenous carboxylate reductase and an exogenous alcohol dehydrogenase, said host further producing 1,6-hexanediol.

40.-43. (canceled)

44. The recombinant host of claim 38, said host further comprising one or more of the following exogenous enzymes: a glutamate synthase, a 2-oxoglutarate decarboxylase, a branch-chain decarboxylase, a glutamate decarboxylase, a ω-transaminase, a CoA-ligase, a CoA-transferase, and an alcohol dehydrogenase.

45. A bio-derived product, bio-based product or fermentation-derived product, wherein said product comprises:
i. a composition comprising at least one bio-derived, bio-based or fermentation-derived compound or salt thereof produced according to claim 1 or claim 9, or any one of FIGS. 1-5, or any combination thereof,
ii. a bio-derived, bio-based or fermentation-derived polymer comprising the bio-derived, bio-based or fermentation-derived composition or compound of i., or any combination thereof,
iii. a bio-derived, bio-based or fermentation-derived resin comprising the bio-derived, bio-based or fermentation-derived compound or bio-derived, bio-based or fermentation-derived composition of i. or any combination thereof or the bio-derived, bio-based or fermentation-derived polymer of ii. or any combination thereof,
iv. a molded substance obtained by molding the bio-derived, bio-based or fermentation-derived polymer of ii. or the bio-derived, bio-based or fermentation-derived resin of iii., or any combination thereof,
v. a bio-derived, bio-based or fermentation-derived formulation comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., or bio-derived, bio-based or fermentation-derived molded substance of iv, or any combination thereof, or vi. a bio-derived, bio-based or fermentation-derived semi-solid or a non-semi-solid stream, comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., bio-derived, bio-based or fermentation-derived formulation of v., or bio-derived, bio-based or fermentation-derived molded substance of iv., or any combination thereof.

46. A non-naturally occurring organism comprising at least one exogenous nucleic acid encoding at least one polypeptide having the activity of at least one enzyme depicted in any one of FIGS. 1 to 5.

47. A non-naturally occurring biochemical network comprising a 4-hydroxybutyryl-CoA, an exogenous nucleic acid encoding a polypeptide having the activity of a β-ketothiolase classified under EC. 2.3.1, and a 3-oxo-6-hydroxyhexanoyl-CoA.

48. A nucleic acid construct or expression vector comprising
(a) a polynucleotide encoding a polypeptide having 3-ketothiolase activity, wherein the polynucleotide is operably linked to one or more heterologous control sequences that direct production of the polypeptide and wherein the polypeptide having β-ketothiolase activity is selected from the group consisting of: (a) a polypeptide having at least 70% sequence identity to the polypeptide of SEQ ID NOs: 1, 13 or 14;
(b) a polynucleotide encoding a polypeptide having ω-transaminase activity, wherein the polynucleotide is operably linked to one or more heterologous control sequences that direct production of the polypeptide and wherein the polypeptide having ω-transaminase activity is selected from the group consisting of: (a) a polypeptide having at least 70% sequence identity to the polypeptide of SEQ ID NOs: 7-12;
(c) a polynucleotide encoding a polypeptide having carboxylate reductase activity, wherein the polynucleotide is operably linked to one or more heterologous control sequences that direct production of the polypeptide and wherein the polypeptide having carboxylate reductase activity is selected from the group consisting of: (a) a polypeptide having at least 70% sequence identity to the polypeptide of SEQ ID NOs: 2-6; or
(d) a polynucleotide encoding a polypeptide having 3-hydroxyacyl-CoA dehydrogenase, 3-oxoacyl-CoA reductase, enoyl-CoA hydratase, trans-2-enoyl-CoA reductase, thioesterase CoA transferase, monooxygenase, alcohol dehydrogenase, 4-hydroxybutanoate dehydrogenase, 5-hydroxyvalerate dehydrogenase, 6-hydroxyhexanoate dehydrogenase, 7-oxoheptanoate dehydrogenase, 6-oxohexanoate dehydrogenase, 5-oxovalerate dehydrogenase, aldehyde dehydrogenase, 6-hydroxyhexanoate dehydrogenase, 5-hydroxypentanoate dehydrogenase, ω-transaminase, amidohydrolase, glutamate synthase, 2-oxoglutarate decarboxylase, branch chain decarboxylase, glutamate decarboxylase, ω-transaminase, CoA transferase, or CoA ligase activity.

49. A composition comprising the nucleic acid construct or expression vector of claim 48.


SEQUENCE LISTING (17 sequences)

SEQ ID NO: 1 - protein, 394 aa, Cupriavidus necator
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI

SEQ ID NO: 2 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 3 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 4 - protein, 1148 aa, Segniliparus rugosus
MGDGEERAKRFFQRIGELSATDPQFAAAAPDPAVVEAVSDPSLSFTRYLDTLMRGYAERPALAHRVGAGYETISYGELWARVGAIAAAWQADGLAPGDFVATVGFTSPDYVAVDLAAARSGLVSVPLQAGASLAQLVGILEETEPKVLAASASSLEGAVACALAAPSVQRLVVFDLRGPDASESAADERRGALADAEEQLARAGRAVVVETLADLAARGEALPEAPLFEPAEGEDPLALLIYTSGSTGAPKGAMYSQRLVSQLWGRTPVVPGMPNISLHYMPLSHSYGRAVLAGALSAGGTAHFTANSDLSTLFEDIALARPTFLALVPRVCEMLFQESQRGQDVAELRERVLGGRLLVAVCGSAPLSPEMRAFMEEVLGFPLLDGYGSTEALGVMRNGIIQRPPVIDYKLVDVPELGYRTTDKPYPRGELCIRSTSLISGYYKRPEITAEVFDAQGYYKTGDVMAEIAPDHLVYVDRSKNVLKLSQGEFVAVAKLEAAYGTSPYVKQIFVYGNSERSFLLAVVVPNAEVLGARDQEEAKPLIAASLQKIAKEAGLQSYEVPRDFLIETEPFTTQNGLLSEVGKLLRPKLKARYGEALEARYDEIAHGQADELRALRDGAGQRPVVETVVRAAVAISGSEGAEVGPEANFADLGGDSLSALSLANLLHDVFEVEVPVRIIIGPTASLAGIAKHIEAERAGASAPTAASVHGAGATRIRASELTLEKFLPEDLLAAAKGLPAADQVRTVLLTGANGWLGRFLALEQLERLARSGQDGGKLICLVRGKDAAAARRRIEETLGTDPALAARFAELAEGRLEVVPGDVGEPKFGLDDAAWDRLAEEVDVIVHPAALVNHVLPYHQLFGPNVVGTAEIIRLAITAKRKPVTYLSTVAVAAGVEPSSFEEDGDIRAVVPERPLGDGYANGYGNSKWAGEVLLREAHELVGLPVAVFRSDMILAHTRYTGQLNVPDQFTRLVLSLLATGIAPKSFYQQGAAGERQRAHYDGIPVDFTAEAITTLGAEPSWFDGGAGFRSFDVFNPHHDGVGLDEFVDWLIEAGHPISRIDDHKEWFARFETAVRGLPEAQRQHSLLPLLRAYSFPHPPVDGSVYPTGKFQGAVKAAQVGSDHDVPHLGKALIVKYADDLKALGLL

SEQ ID NO: 5 - protein, 1185 aa, Mycobacterium massiliense
MTNETNPQQEQLSRRIESLRESDPQFRAAQPDPAVAEQVLRPGLHLSEAIAALMTGYAERPALGERARELVIDQDGRTTLRLLPRFDTTTYGELWSRTTSVAAAWHHDATHPVKAGDLVATLGFTSIDYTVLDLAIMILGGVAVPLQTSAPASQWTTILAEAEPNTLAVSIELIGAAMESVRATPSIKQVVVFDYTPEVDDQREAFEAASTQLAGTGIALETLDAVIARGAALPAAPLYAPSAGDDPLALLIYTSGSTGAPKGAMHSENIVRRWWIREDVMAGTENLPMIGLNFMPMSHIMGRGTLTSTLSTGGTGYFAASSDMSTLFEDMELIRPTALALVPRVCDMVFQRFQTEVDRRLASGDTASAEAVAAEVKADIRDNLFGGRVSAVMVGSAPLSEELGEFIESCFELNLTDGYGSTEAGMVFRDGIVQRPPVIDYKLVDVPELGYFSTDKPHPRGELLLKTDGMFLGYYKRPEVTASVFDADGFYMTGDIVAELAHDNIEIIDRRNNVLKLSQGEFVAVATLEAEYANSPVVHQIYVYGSSERSYLLAVVVPTPEAVAAAKGDAAALKTTIADSLQDIAKEIQLQSYEVPRDFIIEPQPFTQGNGLLTGIAKLARPNLKAHYGPRLEQMYAEIAEQQAAELRALHGVDPDKPALETVLKAAQALLGVSSAELAADAHFTDLGGDSLSALSFSDLLRDIFAVEVPVGVIVSAANDLGGVAKFVDEQRHSGGTRPTAETVHGAGHTEIRAADLTLDKFIDEATLHAAPSLPKAAGIPHTVLLTGSNGYLGHYLALEWLERLDKTDGKLIVIVRGKNAEAAYGRLEEAFDTGDTELLAHFRSLADKHLEVLAGDIGDPNLGLDADTWQRLADTVDVIVHPAALVNHVLPYNQLFGPNVVGTAEIIKLAITTKIKPVTYLSTVAVAAYVDPTTFDEESDIRLISAVRPIDDGYANGYGNAKWAGEVLLREAHDLCGLPVAVFRSDMILAHSRYTGQLNVPDQFTRLILSLIATGIAPGSFYQAQTTGERPLAHYDGLPGDFTAEAITTLGTQVPEGSEGFVTYDCVNPHADGISLDNFVDWLIEAGYPIARIDNYTEWFTRFDTAIRGLSEKQKQHSLLPLLHAFEQPSAAENHGVVPAKRFQHAVQAAGIGPVGQDGTTDIPHLSRRLIVKYAKDLEQLGLL

SEQ ID NO: 6 - protein, 1186 aa, Segniliparus rotundus
MTQSHTQGPQASAAHSRLARRAAELLATDPQAAATLPDPEVVRQATRPGLRLAERVDAILSGYADRPALGQRSFQTVKDPITGRSSVELLPTFDTITYRELRERATAIASDLAHHPQAPAKPGDFLASIGFISVDYVAIDIAGVFAGLTAVPLQTGATLATLTAITAETAPTLFAASIEHLPTAVDAVLATPSVRRLLVFDYRAGSDEDREAVEAAKRKIADAGSSVLVDVLDEVIARGKSAPKAPLPPATDAGDDSLSLLIYTSGSTGTPKGAMYPERNVAHFWGGVWAAAFDEDAAPPVPAINITFLPLSHVASRLSLMPTLARGGLMHFVAKSDLSTLFEDLKLARPTNLFLVPRVVEMLYQHYQSELDRRGVQDGTREAEAVKDDLRTGLLGGRILTAGFGSAPLSAELAGFIESLLQIHLVDGYGSTEAGPVWRDGYLVKPPVTDYKLIDVPELGYFSTDSPHPRGELAIKTQTILPGYYKRPETTAEVFDEDGFYLTGDVVAQIGPEQFAYVDRRKNVLKLSQGEFVTLAKLEAAYSSSPLVRQLFVYGSSERSYLLAVIVPTPDALKKFGVGEAAKAALGESLQKIARDEGLQSYEVPRDFIIETDPFTVENGLLSDARKSLRPKLKEHYGERLEAMYKELADGQANELRDIRRGVQQRPTLETVRRAAAAMLGASAAEIKPDAHFTDLGGDSLSALTFSNFLHDLFEVDVPVGVIVSAANTLGSVAEHIDAQLAGGRARPTFATVHGKGSTTIKASDLTLDKFIDEQTLEAAKHLPKPADPPRTVLLTGANGWLGRFLALEWLERLAPAGGKLITIVRGKDAAQAKARLDAAYESGDPKLAGHYQDLAATTLEVLAGDFSEPRLGLDEATWNRLADEVDFISHPGALVNHVLPYNQLFGPNVAGVAEIIKLAITTRIKPVTYLSTVAVAAGVEPSALDEDGDIRTVSAERSVDEGYANGYGNSKWGGEVLLREAHDRTGLPVRVFRSDMILAHQKYTGQVNATDQFTRLVQSLLATGLAPKSFYELDAQGNRQRAHYDGIPVDFTAESITTLGGDGLEGYRSYNVFNPHRDGVGLDEFVDWLIEAGHPITRIDDYDQWLSRFETSLRGLPESKRQASVLPLLHAFARPGPAVDGSPFRNTVFRTDVQKAKIGAEHDIPHLGKALVLKYADDIKQLGLL

SEQ ID NO: 7 - protein, 459 aa, Chromobacterium violaceum
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 8 - protein, 468 aa, Pseudomonas aeruginosa
MNARLHATSPLGDADLVRADQAHYMHGYHVFDDHRVNGSLNIAAGDGAYIYDTAGNRYLDAVGGMWCTNIGLGREEMARTVAEQTRLLAYSNPFCDMANPRAIELCRKLAELAPGDLDHVFLTTGGSTAVDTAIRLMHYYQNCRGKRAKKHVITRINAYHGSTFLGMSLGGKSADRPAEFDFLDERIHHLACPYYYRAPEGLGEAEFLDGLVDEFERKILELGADRVGAFISEPVFGSGGVIVPPAGYHRRMWELCQRYDVLYISDEVVTSFGRLGHFFASQAVFGVQPDIILTAKGLTSGYQPLGACIFSRRIWEVIAEPDKGRCFSHGFTYSGHPVACAAALKNIEIIEREGLLAHADEVGRYFEERLQSLRDLPIVGDVRGMRFMACVEFVADKASKALFPESLNIGEWVHLRAQKRGLLVRPIVHLNVMSPPLILTREQVDTVVRVLRESIEETVEDLVRAGHR

SEQ ID NO: 9 - protein, 454 aa, Pseudomonas syringae
MSANNPQTLEWQALSSEHHLAPFSDYKQLKEKGPRIITRAEGVYLWDSEGNKILDGMSGLWCVAIGYGREELADAASKQMRELPYYNLFFQTAHPPVLELAKAISDIAPEGMNHVFFTGSGSEGNDTMLRMVRHYWALKGQPNKKTIISRVNGYHGSTVAGASLGGMTYMHEQGDLPIPGVVHIPQPYWFGEGGDMTPDEFGIWAAEQLEKKILELGVENVGAFIAEPIQGAGGVIVPPDSYWPKIKEILSRYDILFAADEVICGFGRTSEWFGSDFYGLRPDMMTIAKGLTSGYVPMGGLIVRDEIVAVLNEGGDFNHGFTYSGHPVAAAVALENIRILREEKIVERVRSETAPYLQKRLRELSDHPLVGEVRGVGLLGAIELVKDKTTRERYTDKGAGMICRTFCFDNGLIMRAVGDTMIIAPPLVISFAQIDELVEKARTCLDLTLAVLQG

SEQ ID NO: 10 - protein, 467 aa, Rhodobacter sphaeroides
MTRNDATNAAGAVGAAMRDHILLPAQEMAKLGKSAQPVLTHAEGIYVHTEDGRRLIDGPAGMWCAQVGYGRREIVDAMAHQAMVLPYASPWYMATSPAARLAEKIATLTPGDLNRIFFTTGGSTAVDSALRFSEFYNNVLGRPQKKRIIVRYDGYHGSTALTAACTGRTGNWPNFDIAQDRISFLSSPNPRHAGNRSQEAFLDDLVQEFEDRIESLGPDTIAAFLAEPILASGGVIIPPAGYHARFKAICEKHDILYISDEVVTGFGRCGEWFASEKVFGVVPDIITFAKGVTSGYVPLGGLAISEAVLARISGENAKGSWFTNGYTYSNQPVACAAALANIELMEREGIVDQAREMADYFAAALASLRDLPGVAETRSVGLVGCVQCLLDPTRADGTAEDKAFTLKIDERCFELGLIVRPLGDLCVISPPLIISRAQIDEMVAIMRQAITEVSAAHGLTAKEPAAV

SEQ ID NO: 11 - protein, 459 aa, Escherichia coli
MNRLPSSASALACSAHALNLIEKRTLDHEEMKALNREVIEYFKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRTALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 12 - protein, 453 aa, Vibrio fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 13 - protein, 401 aa, Escherichia coli
MREAFICDGIRTPIGRYGGALSSVRADDLAAIPLRELLVRNPRLDAECIDDVILGCANQAGEDNRNVARMATLLAGLPQSVSGTTINRLCGSGLDALGFAARAIKAGDGDLLIAGGVESMSRAPFVMGKAASAFSRQAEMFDTTIGWRFVNPLMAQQFGTDSMPETAENVAELLKISREDQDSFALRSQQRTAKAQSSGILAEEIVPVVLKNKKGVVTEIQHDEHLRPETTLEQLRGLKAPFRANGVITAGNASGVNDGAAALIIASEQMAAAQGLTPRARIVAMATAGVEPRLMGLGPVPATRRVLERAGLSIHDMDVIELNEAFAAQALGVLRELGLPDDAPHVNPNGGAIALGHPLGMSGARLALAASHELHRRNGRYALCTMCIGVGQGIAMILERV

SEQ ID NO: 14 - protein, 438 aa, Clostridium aminobutyricum
MDWKKIYEDRTCTADEAVKSIKSGDRVLFAHCVAEPPVLVEAMVANAAAYKNVTVSHMVTLGKGEYSKPEYKENFTFEGWFTSPSTRGSIAEGHGQFVPVFFHEVPSLIRKDIFHVDVFMVMVSPPDHNGFCCVGVSSDYTMQAIKSAKIVLAEVNDQVPVVYGDTFVHVSEIDKFVETSHPLPEIGLPKIGEVEAAIGKHCASLIEDGSTLQLGIGAIPDAVLSQLKDKKHLGIHSEMISDGVVDLYEAGVIDCSQKSIDKGKMAITFLMGTKRLYDFAANNPKVELKPVDYINHPSVVAQCSKMVCINACLQVDFMGQIVSDSIGTKQFSGVGGQVDFVRGASMSIDGKGKAIIAMPSVAKKKDGSMISKIVPFIDHGAAVTTSRNDADYVVTEYGIAEMKGKSLQDRARALINIAHPDFKDELKAEFEKRFNAAF

SEQ ID NO: 15 - protein, 397 aa, Treponema denticola
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 16 - protein, 539 aa, Euglena gracilis
MSCPASPSAAVVSAGALCLCVATVLLATGSNPTALSTASTRSPTSLVRGVDRGLMRPTTAAALTTMREVPQMAEGFSGEATSAWAAAGPQWAAPLVAAASSALALWWWAARRSVRRPLAALAELPTAVTHLAPPMAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 17 - protein, 550 aa, Salmonella typhimurium
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG